(19) United States
(12) Patent Application Publication
(10) Pub. No.: US 2011/0171665 A1
(43) Pub. Date: Jul. 14, 2011

(54) P-SELECTIN ASSOCIATED WITH EOSINOPHILS AS A MARKER FOR ASTHMA AND CORRELATING WITH B-1 INTEGRIN ACTIVATION

(75) Inventors: Mats W. Johansson, Madison, WI (US); Deane F. Mosher, Madison, WI (US)

(73) Assignee: WISCONSIN ALUMNI RESEARCH FOUNDATION, Madison, WI (US)

(21) Appl. No.: 13/074,822

(22) Filed: Mar. 29, 2011

Related U.S. Application Data
(63) Continuation of application No. 12/129,317, filed on May 29, 2008, now abandoned.
(60) Provisional application No. 60/940,602, filed on May 29, 2007.

(51) Int. Cl. G01N 33/53; C12Q 1/06; G01N 33/68

(52) U.S. Cl. 435/7.24; 435/39; 436/86; 435/7.92; 435/7.21

(57) ABSTRACT

Methods are provided for the detection of P-selectin associated with eosinophils and for the use of P-selectin as a biological marker for asthma. In one embodiment, the present invention relates to methods for detecting P-selectin in a sample containing eosinophils and quantifying the total number of eosinophils. In another embodiment, the present invention relates a method for determining the proportion of eosinophils that are P-selectin positive and positive for at least partially activated β-1 integrin. In yet another embodiment, the present invention relates to kits for the detection of P-selectin and for the detection of eosinophils that are both P-selectin positive and positive for at least partially activated β-1 integrin. In still yet another embodiment, the invention relates to a method for monitoring a biological condition.

### CROSS-REFERENCE TO RELATED APPLICATIONS

This application claims priority to provisional patent application Ser. No. 60/940,602, which was filed on May 29, 2007, and is incorporated herein by reference.

### REFERENCE TO GOVERNMENT GRANT

This invention was made with United States government support awarded by the following agency: NIH HL056396; NIH HL080412; NIH HL069116; NIH HL088594; Clinical and Translational Science Award Grant 1UL 1RR025011; and General Clinical Research Center grant M01 RR03186 from NIH. The United States has certain rights in this invention

### BIBLIOGRAPHY

Complete bibliographic citations of the references referred to herein by authors' names can be found in the Bibliography section, immediately following the Examples. The references listed in the bibliography are incorporated by reference into the application in their entireties.

### FIELD OF THE INVENTION

The present invention relates to the use of P-selectin as a marker for asthma or other biological condition, for example a respiratory disorder. The present invention also relates to methods for monitoring asthma using P-selectin. The present invention also relates to methods for monitoring asthma comprising detecting eosinophils that are positive for P-selectin and positive for at least partially activated β-1 integrin. In addition, the present invention relates to methods of treating asthma using P-selectin-triggered eosinophil β₁ activation as a potential therapeutic target.

### DESCRIPTION OF THE RELATED ART

Asthma is a chronic disease affecting over 20 million Americans. Asthma is characterized by (i) bronchoconstriction, (ii) excessive mucus production, and (iii) inflammation and swelling of airways. These conditions cause widespread and variable airflow obstruction thereby making it difficult for the asthma sufferer to breathe. Asthma further includes acute episodes or attacks of additional airway narrowing via contraction of hyper-responsive airway smooth muscle.

Currently, a number of tests are available for diagnosing a person with asthma. However, there are limited tests for monitoring treatment efficacy of the disease and features of the disease. Collection and analysis of the sputum produced during an asthmatic attack is one method of measuring disease severity. However, this method is inconsistent and tedious. Another method for analyzing airway inflammation is through measurement of exhaled nitric oxide (NO), which is produced as a result of an increase in reactive oxygen species resulting from the body's inflammatory response. The NO exhalation test, however, is rather inaccurate; the flow rate with which the patient exhales can affect NO concentration. Also, due to the high baseline airway inflammation of a person suffering from asthma, NO levels may be high in patients in which the disease is under control.

Asthma is characterized by eosinophilic inflammation. Eosinophils are a family of white blood cells that are attracted to areas where foreign substances enter the body. Eosinophils release toxic substances to kill the invaders. Recent observations have indicated that the presence of sputum eosinophils is an indicator of asthma instability or likelihood of an asthma exacerbation. Studies also suggest that the recruitment of eosinophils to the airway may be a key step in the development of an asthma exacerbation. Interaction between α₄β₁ integrin on eosinophils and vascular cell adhesion molecule-1 (VCAM-1) on cytokine-stimulated endothelium is believed to be essential for eosinophils to move from the bloodstream into tissue.

Eosinophil adhesion receptors of the integrin family and their cognate ligands in the lung are indicated as important participants in eosinophil recruitment. (Giembych M A, et al.; Seminario M C, et al.) Integrin-mediated adhesion is a function of ligand density, cell-surface integrin density, and integrin activation state. (Palecek S P, et al.). α₄β₁ on various cell types exists in several different conformations or activation states, which can be probed with activation-sensitive anti-β₁ monoclonal antibodies (mAbs). (Humphries M J, et al.) Unlike many other integrin-ligand pairings, α₄β₁ mediates a certain degree of eosinophil adhesion to VCAM-1 in the absence of stimulation by cytokines or other soluble factors, and therefore does not require so-called “inside-out” activation of the integrin for a baseline level of adhesion. (Barthel S R, et al.; Weller P F, et al.) The preferential induction of the ligand VCAM-1 is one mechanism causing selective eosinophil adherence and movement from the circulation. Results on eosinophils in blood from mild asthmatics undergoing ICS withdrawal indicate that β₁ activation state, as assessed by expression of the epitope for mAb N29, varies and may be elevated, which would be expected to be linked to enhanced interaction of eosinophil α₄β₁ with VCAM-1. (Johansson M W, et al., 2006) This may be a second mechanism contributing to the regulation of eosinophil adherence and extravasation.

The epitope for N29 maps to amino acid residue No. 4, a glutamic acid, near the amino (N) terminus of the N-terminal plexin, semaphorin, and integrin (PSI) domain of the mature β₁ subunit. (Wilkins J A, et al.; Mould A P, et al., 2005) Another activation-sensitive anti-β₁ mAb, 8E3, recognizes the same amino acid residue. (Coe A P, et al.; Mould A P, et al., 2005) Analysis of the crystal and electron microscopic structures of β₃ integrins and mapping of anti-β₁ mAbs onto the structures indicates that the integrin conformation that is bent or un-extended places the PSI domain close to the α subunit, thereby making the N29 epitope likely inaccessible and having no or low affinity for ligand. (Humphries M J et al.; Mould A P, et al., 2005) In the extended, unbent, but not fully open conformation, which likely has low-intermediate affinity, the PSI domain is located under the ligand-binding “head” of the integrin heterodimer, and the N29 epitope is likely accessible; thus N29 is believed to detect unbending or extension. In the fully open, conformation, which likely has high affinity for ligands, the hybrid and PSI domains of the β₁ subunit are “swung out” and point outward, likely allowing accessibility to mAb HUTS-21, whose epitope is in the hybrid domain and believed to detect the “swing-out.” (Luque A, et al.) In the high-affinity conformation, the epitope for mAb 9EG7, which is in the epidermal growth factor (EGF)-like domains of the β₁ “leg,” also is likely accessible; and 9EG7 likely detects integrin “leg separation” of the membrane-proximal domains of the α and β subunits. Another β₁ mAb, 12G10, recognizes an activated conformation of the ligand-binding I domain, which is believed to be coupled to hybrid domain “swing-out.” (Mould A P, et al., 1995)

There are numerous ligands on eosinophils including the P-selectin glycoprotein ligand, which is a disulfide-bonded, homodimeric mucin (˜250 kDa) that binds to P-selectin on platelets and endothelial cells during the initial steps in inflammation. P-selectin is normally sequestered in a granules of platelets and Weibel-Palade bodies of endothelial cells and translocated to the surface in response to thrombogenic agents, such as histamine, thrombin, and Th1 and Th2 mediators. (Andre P) Other names for P-selectin include CD62P, Granule Membrane Protein 140 (GMP-140), and Platelet Activation-Dependent Granule to External Membrane Protein (PADGEM). Addition of soluble P-selectin to monocytes increased β₁ activation state, as judged by induction of the HUTS-21 epitope, and promoted monocyte adhesion to and arrest on VCAM-1. (Yago T, et al.; da Costa Martins P A, et al.) Serum P-selectin concentration was decreased in mild asthmatics after inhalation of the β agonist salbutamol and was proposed to be a marker in mild asthma. (Sjosward K N, et al.)

However, it is unknown what stimulus or stimuli cause(s) a change or changes in the β₁ conformation and elevated activation state on eosinophils. Determining the factors responsible for sustained increased β-1 integrin activation should allow a better understanding of the observed phenomenon of β-1 activation in asthmatic patients.

In view of the foregoing, it would be desirable to provide more reliable methods of detecting markers related to asthma control. It would also be desirable to provide kits for practicing these methods. Furthermore, it would be desirable to identify biological pathways that can be used as therapeutic targets.

### SUMMARY OF THE INVENTION

The invention, which is defined by the claims set out at the end of this disclosure, is intended to solve at least some of the problems noted above. In one embodiment, the present invention provides a method for detecting P-selectin in a sample. In another embodiment, the invention relates to a method for detecting P-selectin associated with eosinophils in a sample, and quantifying the total number of eosinophils in the sample.

In yet another embodiment, a sample can be obtained from a source including but not limited to whole blood, serum, plasma and sputum.

In still yet another embodiment, P-selectin detected by the methods of the invention can be associated with a cell, associated with an eosinophil, associated with a neutrophil, associated with a monocyte, free in plasma, free in serum and free in whole blood.

In another embodiment, the present invention relates to a method comprising using P-selectin as a biological marker for asthma or other biological condition, for example a respiratory disorder. A sample is obtained from a subject. In one embodiment, the sample is whole blood. An amount of P-selectin associated with eosinophils can be detected. The total number of eosinophils in the sample can be quantified. In still yet another embodiment, the invention relates to a method comprising detecting P-selectin in a sample and determining relative lung function based on the amount of P-selectin detected in the sample.

The step of detecting can comprise conducting an assay to determine binding of P-selectin to a binding partner of P-selectin. The assay can be performed by flow cytometry, an ELISA assay, and an ELISA-like assay. However, other assays can also be used.

The binding partner can be an antibody that binds to P-selectin. An example of such an antibody is the monoclonal antibody AC1.2. The total number of eosinophils in the sample can be quantified using an eosinophil peroxidase assay or other assays.

Additional steps also can be included in the method. These steps can include determining a baseline of P-selectin in a baseline sample from the subject. An additional sample can be obtained from the subject, wherein the amount of P-selectin can be determined. The baseline of P-selectin can be compared to the amount of P-selectin in the additional sample. P-selectin can be associated with eosinophils. The additional sample can be obtained prior to an asthmatic attack, during an asthmatic attack, and after an asthmatic attack. In yet another embodiment, the additional sample can be obtained prior to the onset of an episode of a respiratory disorder, during an episode of a respiratory disorder, and after an episode of a respiratory disorder.

In another embodiment, the present invention provides a method for monitoring or detecting asthma comprising detecting the proportion of eosinophils that are P-selectin positive and positive for at least partially activated β-1 integrin. In one embodiment, the step of detecting the eosinophils comprises flow cytometry, including but not limited to a dual labeling flow cytometry procedure. In still yet another embodiment, the method of detecting at least partially activated β-1 integrin comprises conducting an assay to determine binding of at least partially activated β-1 integrin to a binding partner of activated β-1 integrin. Any suitable assay can be used to determine the binding of at least partially activated β-1 integrin including but not limited to flow cytometry, an ELISA assay, and an ELISA-type assay. The binding partner can be any suitable reagent including but not limited to a polyclonal antibody, a monoclonal antibody (mAb), mAb N29, mAb 8E3, mAb HUTS-21, and mAb 9EG7.

In still yet another embodiment, the invention relates to a method for monitoring asthma, the method comprising detecting an amount of P-selectin in a sample from a subject; and comparing the amount of P-selectin to a baseline amount, wherein an increase or decrease in the amount of P-selectin is indicative of a change in asthmatic condition. P-selectin can be associated with eosinophils or in plasma. In still yet another embodiment, the method can further comprise determining a baseline of at least partially activated β-1 integrin in the same baseline sample or a different baseline sample from the subject. An additional sample can be obtained from the subject, wherein the amount of at least partially activated β-1 integrin can be determined. The baseline of at least partially activated β-1 integrin can be compared to the amount of at least partially activated β-1 integrin in the additional sample.

In yet another embodiment, the present invention relates to a method of monitoring a biological condition, the method comprising detecting an amount of P-selectin in a sample from a subject; detecting an amount of β-1 integrin activation; and comparing the amount of P-selectin and β-1 integrin activation to a baseline amount, wherein an increase or decrease in the amount of P-selectin and β-1 integrin activation is indicative of a change in biological condition. The biological condition can be a respiratory disorder including but not limited to acute bronchitis, asthma, bacterial pneumonia, chemical pneumonia, bronchoscopy, chronic bronchitis, collapsed lung, emphysema, hyperventilation, pleurisy, pulmonary edema, pulmonary embolism, and viral pneumonia.

In yet another embodiment, the present invention provides a method for identifying agents that inhibit or impede P-selectin mediated activation of β-1 integrin.

In still another embodiment, the present invention provides kits for determining the amount of P-selectin in a sample. The kit includes a positive control including cells that express P-selectin and a negative control including cells lacking P-selectin. The kit also can include an anti-P-selectin antibody that binds to P-selectin. Such an antibody can be monoclonal antibody AC1.2. The kit also may include a secondary antibody.

In addition, the kit may also include an antibody or antibodies to detect the activation state of β-1 integrin. Various antibodies that recognize different states of activation of β-1 may be provided in the kit including but not limited to mAb N-29, mAb 8E3, mAb HUTS-21, mAb 9EG7, and mAb 12G10. Instructions may also be included with the kit, which may provide information on incubation times, dilution factors for various antibodies, appropriate secondary antibodies, and general information on how to perform the assays.

### BRIEF DESCRIPTION OF THE DRAWINGS

Preferred exemplary embodiments of the invention are illustrated in the accompanying drawings, in which like reference numerals represent like parts throughout and in which:

FIG. 1A and FIG. 1B report primary flow cytometric data on eosinophils in whole blood from mild/moderate asthmatics during and after an upper respiratory tract infection. FIG. 1A reports the correlation between β₁ integrin activation state, as measured by mAb N29, and associated P-selectin. FIG. 1B demonstrates that Eosinophil N29 reactivity does not correlate with eosinophil PSGL-1 expression. gMCF=geometric mean channel fluorescence, P=probability, rₛ=Spearman rank correlation coefficient.

FIG. 2A and FIG. 2B report flow cytometric data. FIG. 2A reports the inverse correlation between associated P-selectin and FEV₁ in whole blood in mild/moderate asthmatics during and after an upper respiratory tract infection. No correlation was observed between PSGL-1 expression and FEV₁ (FIG. 2B). gMCF=geometric mean channel fluorescence, P=probability, rₛ=Spearman rank correlation coefficient.

FIG. 3A-FIG. 3E report flow cytometric data. FIG. 3A reports the division of two eosinophil populations, one with a low (white) and one with a high (shaded) level of associated P-selectin. FIG. 3B reports the isotype control versus associated P-selectin on eosinophils. FIG. 3C reports the expression of β₁ activation-sensitive epitope, as measured by mAb N29, versus associated P-selectin on eosinophils. FIG. 3D reports the expression of β-1 activation sensitive epitope, as measured by mAb 12G10, versus associated P-selectin on eosinophils. FIG. 3E reports PSGL-1 expression versus associated P-selectin on eosinophils.

FIG. 3F-FIG. 3H are bar graphs representing specific expression (±standard deviation) of β₁ epitopes and PSGL-1 in P-selectin-low and P-selectin-high populations of eosinophils (FIG. 3F), neutrophils (FIG. 3G), and monocytes (FIG. 3H) (n=3, except n=2 for mAb HUTS-21 and mAb 9EG7). *P<0.05 versus P-selectin-low population, **P<0.01 versus P-selectin-low population (t test). PS=P-selectin.

FIG. 4A-FIG. 4D are graphs reporting the effect of added soluble P-selectin on mAb N29 epitope expression. FIG. 4A is a line graph representing mAb N29 epitope expression of eosinophils in whole blood after pre-incubation for 1 h with P-selectin (0.1 μg/ml) (thick line) or without P-selectin (normal line), or isotype control (thin line). FIG. 4B is a line graph representing mAb N29 epitope expression in eosinophils in whole blood (filled symbols) or purified eosinophils (empty symbols) with different concentrations of P-selectin. FIG. 4C is a bar graph representing mAb N29 epitope expression in leukocytes in whole blood without added soluble P-selectin. FIG. 4D is a bar graph representing mAb N29 epitope expression in leukocytes without added soluble P-selectin (white bars) or with added soluble P-selectin (1 μg/ml) (shaded bars) from the same donor.

FIG. 5A and FIG. 5B are line graphs depicting N29 epitope expression on eosinophils and eosinophilic leukemic EoL-3 cells in suspension before and after adhesion to VCAM-1. FIG. 5A is a line graph representing purified eosinophils and FIG. 5B is a line graph representing EoL-3 cells: original cell population before adhesion to VCAM-1 (normal line), cells non-adherent to a substrate coated with VCAM-1 (thick line) (10 μg/ml for eosinophils, 1 μg/ml for EoL-3 cells), isotype control (thin line).

FIG. 6A-FIG. 6D report primary flow cytometric data on eosinophils in whole blood obtained from participants in the multi-center Severe Asthma Research Program (SARP). FIG. 6A reports the correlation between expression of the epitope for activation-sensitive β₁ integrin mAb N29 and associated P-selectin. FIG. 6B reports the correlation between expression of the epitope for activation-sensitive β₁ integrin mAb 12G10 and associated P-selectin. FIG. 6C reports the correlation between expression of the epitope for activation-sensitive β₁ integrin mAb HUTS-21 and associated P-selectin. FIG. 6D reports the correlation between expression of the epitope for activation-sensitive β₁ integrin mAb 9EG7 and associated P-selectin.

It is to be understood that the invention is not limited in its application to the details of construction and the arrangement of the components set forth in the following description of embodiments of the invention or illustrated in the drawings. The invention is capable of other embodiments or being practiced or carried out in various ways. Also, it is to be understood that the phraseology and terminology employed herein is for the purpose of description and should not be regarded as limiting.

### DETAILED DESCRIPTION

Abbreviations used herein include the following:

BSA=bovine serum albumin; Eos=eosinophils; ERK=extracellular-regulated kinase; F=female; FEV₁=forced expiratory volume in 1 s; FITC=fluorescein isothiocyanate; gMCF=geometric mean channel fluorescence; ICS=inhaled corticosteroid; Ig=immunoglobulin; IL=interleukin; M=male; mAb=monoclonal antibody; mono=monocytes; n=number of subjects; neuts=neutrophils; P=probability; PBS=phosphate-buffered saline; PC₂₀=provocative concentration of methacholine producing a 20% fall in FEV₁; PE=phycoerythrin; PS=P-selectin; PSGL-1=P-selectin glycoprotein ligand-1; PSI=plexin, semaphorin, and integrin; RANTES=regulated on activation, normal T-cell expressed and secreted; rpm=revolutions per minute; RPMI=Roswell Park Memorial Institute; rₛ=Spearman rank correlation coefficient; TBS=Tris-buffered saline; Th=T helper cell type; VCAM-1=vascular cell adhesion molecule-1; VIAX=Virus-Induced Asthma Exacerbation; SARP=Severe Asthma Research Program.

The term “agent” includes conventional chemical pharmaceutical compounds, as well as nucleic acids including DNA, RNA, anti-sense molecules and hybrid molecules, polypeptides, polypeptide derivates, peptidomimetics, biological agents, antibodies or other molecules with a desired function.

The term “antibody” as used in this invention includes polyclonal and monoclonal antibodies. The term includes intact molecules as well as fragments thereof, such as Fab, F(ab′)₂, and Fv, which are capable of binding the epitopic determinant.

The term “baseline amount” and like terms means the amount of an analyte measured prior to a subsequent measurement and includes but is not limited to an amount of analyte measured from a normal individual, the amount of an analyte measured prior to the onset of disease or pathological condition, the amount of an analyte measured prior to the worsening of a condition and the amount of an analyte measured prior to the improvement of a disease or pathological condition.

The term “biological condition” and like terms means the condition of a subject in a pertinent realm that is under observation, and such realm may include any aspect of the subject capable of being monitored for change in condition, such as health, disease including cancer; trauma; aging; asthma, infection; a respiratory disorder, tissue degeneration; developmental steps; physical fitness; obesity, and mood. As can be seen, a condition in this context may be chronic or acute or simply transient. Moreover, a targeted biological condition may be manifest throughout the organism or population of cells or may be restricted to a specific organ (such as skin, heart, eye or blood), but in either case, the condition may be monitored directly by a sample of the affected population of cells or indirectly by a sample derived elsewhere from the subject. The term “biological condition” includes a “physiological condition”.

The term “subject” means a living, multi-cellular vertebrate organism; a category that includes both human and veterinary subjects for example, mammals, birds and primates.

The term “treatment” refers to any treatment of a pathologic condition in a mammal, particularly a human, and includes: (i) preventing the pathologic condition from occurring in a subject that may be predisposed to the condition but has not yet been diagnosed with the condition and, accordingly, the treatment constitutes prophylactic treatment for the disease condition; (ii) inhibiting the pathologic condition, i.e., arresting its development; (iii) relieving the pathologic condition, i.e., causing regression of the pathologic condition; or (iv) relieving the conditions mediated by the pathologic condition.

The inventors have found that P-selectin, derived from whole blood, plasma, activated platelets and/or endothelial cells, associates with eosinophils. The inventors have found that the eosinophil-associated P-selectin correlates with β-1 integrin activation state and correlated inversely with FEV₁, which is a measure of lung function. The inventors have found that eosinophil-associated P-selectin enhances eosinophil β₁ activation state, thereby stimulating eosinophil arrest on VCAM-1, egress and recruitment to the airway. The inventors have found that P-selectin associated with eosinophils can be used a marker for asthma. The inventors also have found that P-selectin associated with eosinophils can be used in conjunction with the activation state of β-1 integrin as a marker for asthma or other biological condition, for example a respiratory disorder, and that P-selectin triggered eosinophil β-1 integrin activation can be used as a therapeutic target.

The present invention provides a method for detecting P-selectin associated with eosinophils as a marker for asthma or other biological condition, for example a respiratory disorder. In another embodiment, the present invention provides a method for determining disease status of an individual having asthma including mild asthma, moderate asthma and a severe asthma disease state, based on the detection of P-selectin associated with eosinophils. The present invention further provides methods for determining disease status of an individual having asthma based on the correlation of P-selectin associated with eosinophils and at least partially activated β-1 integrin. Furthermore, the present invention provides a method for treating individuals with asthma based on disrupting or preventing the binding of P-selectin to PSGL. In addition, the present invention provides a method of treating individuals with asthma based on disrupting the signaling pathway between P-selectin bound PSGL and activation of β-1 integrin.

Obtaining a Sample Including Eosinophils from a Patient:

A sample including eosinophils, such as whole blood, lung sputum, or any other fluid containing eosinophils, can be obtained from a patient by conventional methods. The methods and kits described herein can be used on patients having a diagnosis of mild, persistent asthma or a different asthma diagnosis. The methods and kits can also be used on patients having natural fluctuations in their airway disease. Samples can be fixed after binding to an antibody such that the subsequent analysis is carried out at a later time. Samples can be collected and stored such that analysis can be performed at a later time.

In one embodiment where whole blood is used, the amount of whole blood is from about 0.3 ml to about 5 ml of whole blood. The eosinophil (eos) counts for a sample widely vary. The eos count for a normal, non-allergic person (“normals”) is typically about 2% of total WBC. For an allergic asthmatic patient deliberately challenged with antigen, the eos count is up to about 19% of total WBC, which are about 15 million/ml. Typically, asthmatics without extra stimulation have an eos count of up to about 15% of WBC, or about 10 million/ml.

Analysis of a Sample for P-Selectin Associated with Eosinophils

The stimulation of endothelial cells or platelets by inflammatory mediators such as histamine, thrombin, or phorbol esters leads to the surface expression of P-selectin on these stimulated cells. Eosinophil associated P-selectin includes but is not limited to eosinophils binding through the PSGL to P-selectin expressed on platelets, eosinophils binding through the PSGL to P-selectin on endothelial cells, eosinophils binding through PSGL to P-selectin derived from plasma, eosinophils binding to P-selectin expressed on platelets, eosinophils binding to P-selectin on endothelial cells, and eosinophils binding to P-selectin derived from plasma.

P-selectin can be used as a marker for monitoring asthma including but not limited to monitoring the occurrence, the progression, the treatment, the remission, and the disease state of asthma. Detection of P-selectin in a sample, including but not limited to a plasma sample provides an assay including a reproducible and an easy to measure analyte. In addition, the detection of P-selectin may be correlated with other molecules associated with activated platelets or endothelial cells, including but not limited to plasma platelet factor 4, 1-thromboglobulin, and thrombospondin-1.

P-selectin can be detected with any assay capable of detecting P-selectin including but not limited to flow cytometry and an immunoassay. For example, in Examples 1-3, flow cytometry is used to detect P-selectin on eosinophils. Flow cytometry can also be used to detect other epitopes present including but not limited to at least partially activated β₁ integrin. In another embodiment, the sample can be analyzed using an enzyme linked immunosorbant assay (ELISA) or an ELISA-type assay. In one embodiment of the ELISA or ELISA-type assay, a substrate, such as wells on a microtiter plate, is coated with an antibody that binds to P-selectin, such as anti-P-selectin AC1.2. Unbound sites on the substrate can be blocked to prevent false positive results. Preferably, the substrate only binds to P-selectin antibody such that no cells bind directly to the substrate or to a “control” surface of the substrate with no P-selectin antibody immobilized on it.

In one embodiment of the assay, the substrate possesses an inert surface. In another embodiment, the substrate can be a so-called “self-assembled monolayer, as is described in, e.g., U.S. Pat. No. 6,849,321 for “Surfaces with gradients in surface topography,” U.S. Pat. No. 6,824,837 for “Liquid crystal switching mechanism,” U.S. Pat. No. 6,821,485 for “Method and structure for microfluidic flow guiding,” U.S. Pat. No. 6,797,463 for “Method and apparatus for detection of microscopic pathogens,” U.S. Pat. No. 6,746,825 for “Guided self-assembly of block copolymer films on interferometrically nanopatterned substrates,” and U.S. Pat. No. 6,486,334 for “Biomembrane mimetic surface coatings,” all of which are incorporated herein by reference in their entireties.

Self-assembling monolayers (and other substrates) can include a molecule or a group to capture the P-selectin antibody. In addition, an antibody that is engineered with a functional moiety can be utilized to bind to the capturing group in the monolayer on the surface of the substrate.

After the substrate is coated with an antibody, a sample such as blood can be applied to the coated substrate. Cells within the sample having P-selectin can be captured (bound) by the antibody. The substrate then can be washed to remove unbound sample. A secondary antibody can be added to bind with, for example, the antigen-antibody complex or the antibody. The secondary antibody can be conjugated to an enzyme or include a detectable label, such as phycoerythrin (PE), FITC, or the like. Where a secondary antibody is coupled to an enzyme, a development reagent, such as a substrate for the enzyme, can be reacted with the enzyme to produce a detectable product, thus indicating a positive reaction. Where a secondary antibody including a detectable label is used, the label is detected, such as with a fluorometer or another suitable instrument known in the art.

The immunoassay can also be accomplished by Western blotting, 2-dimensional SDS-polyacrylamide gel electrophoresis, or other methods known in the art for detection of specific proteins. The skilled artisan will recognize that the instant invention encompasses all such well-known techniques for detection of the proteins.

In one embodiment of the methods described above, the amount of P-selectin also can be determined. A first sample, which can include, for example eosinophils, can be obtained from the subject. A baseline amount of P-selectin expression in the first sample can be determined. A second sample can be obtained from the same subject. The second sample can be taken when, for example, a healthcare provider wants to determine asthma disease status of the subject. An amount of expression of P-selectin in the second sample is determined. The baseline is compared to the amount of expression of P-selectin in the second sample. P-selection can be associated with eosinophils or cell-free.

The steps of determining and detecting can include conducting an assay with a binding partner of P-selectin. The assay can be an immunoassay, as is described herein, which includes the binding partner of P-selectin. The immunoassay can be chosen from at least one of flow cytometry, an ELISA assay, and an ELISA-like assay. The immunoassay also can be performed with a Western blot on cell extracts or other immunoassays known in the art. Antibodies that bind to P-selectin can be used including but not limited to AC1.2, P-selectin (1 E 3) (Santa Cruz Biotechnology, catalog # sc-19672), P-selectin (AK4) (Santa Cruz Biotechnology, catalog # sc-19996), P-selectin (C-20) (Santa Cruz Biotechnology, catalog # sc-6941), P-selectin (CLB/thromb/6) (Santa Cruz Biotechnology, catalog # sc-59769). Monoclonal and polyclonal P-selectin antibodies are available from a variety of commercial sources including but not limited to BioCytex (France), BioLegend (San Diego, Calif.), Cell Sciences (Canton, Mass.), Chemicon (Temecula, Calif.), GeneTex (San Antonio, Tex.) and Lab Vision (Fremont, Calif.).

In another embodiment, P-selectin can be detected by performing an ELISA assay on frozen samples and detecting plasma P-selectin. Any suitable ELISA can be used to detect P-selectin including a commercial ELISA kit, including but not limited to a P-selectin ELISA kit available from Bender Medsystems (Vienna, Austria) or R&D Systems (Minneapolis, Minn.). The samples can be frozen using any suitable method known in the art including but not limited to exposing the sample to liquid nitrogen, dry ice or a combination of dry ice and liquid nitrogen, and flash-freezing the sample. An ELISA assay on frozen samples can be used to determine if soluble plasma P-selectin correlates with eosinophil β-1 integrin activation state and whether it correlates with FEV₁. Detecting plasma P-selectin by ELISA, or any other suitable assay, with frozen samples, may provide a robust analyte or marker. In addition, detecting plasma P-selectin by ELISA would provide an efficient and inexpensive method.

Correlation of the Amount of P-Selectin and at Least Partially Activated β₁ Integrin in Eosinophils

The level of P-selectin associated with circulating eosinophils can be correlated with at least partially activated β-1 integrin. A dual labeling and flow cytometric quantification of the percentage of eosinophils (see Example 1 below) that are positive for at least partially activated β-1 integrin and positive for P-selectin can be used as a sensitive and specific procedure for monitoring asthma, including but not limited to monitoring the occurrence, the progression, the treatment, the remission, and the disease state of asthma.

An amount of at least partially activated β₁ integrin in the eosinophils can be detected using any of the methods described above including but not limited to flow cytometry, an immunoassay, an ELISA, ELISA-type assay, Western blotting, and 2-dimensional SDS polyacrylamide gel electrophoresis. The term at least partially activated β₁ integrin is meant to encompass β₁ integrin conformations selected from the group consisting of the extended, unbent but not fully open conformation; the fully open conformation; the high affinity conformation, a conformation detected by mAb N-29, a conformation detected by mAb 8E3, a conformation detected by mAb HUTS-21, a conformation detected by mAb 9EG7, and a conformation detected by mAb 12G10. Any antibody the recognizes the β-1 integrin activation state can be used in the assays including but not limited to mAb N-29, mAb 8E3, mAb HUTS-21, mAb 9EG7, and mAb 12G10.

In addition, the immunoassay to detect the amount of β₁ integrin activation on eosinophils can be a modification of an eosinophil peroxidase (EPO) assay, as described below. In a modified EPO assay, VCAM-1, which is normally used in the EPO assay, is replaced with an antibody that recognizes the activation state of β-1 integrin.

For the eosinophil peroxide assay for the quantitation of attached eosinophils, a 96 well plate is coated in triplicate with an antibody that recognizes the activation state of β-1 integrin, e.g. mAb N29 (add 100 μl per well of antibody, at an approximate concentration, e.g., 10 μg/ml). The 96 well plate can be either a tissue or a non-tissue culture treated plate. The antibody is allowed to coat for 2 hours or overnight at 37° C.

The antibody is decanted, and wells are blocked with 0.1% gelatin or fetal bovine serum (FBS) (heat-inactivated) for 15 minutes. Blocker is decanted, and 100 μl of whole blood (undiluted or diluted in HBSS+Ca²⁺ containing 0.2% BSA) or 10,000 eosinophils resuspended in HBSS+Ca²⁺ containing 0.2% BSA per well (100 μl) are added. The plate is incubated at 37° C., in CO₂ incubator for 60 minutes. The original eosinophil stock solution is saved for 100% control stock and is also incubated in an incubator.

During incubation, OPD substrate solution (omitting OPD itself, which is kept on ice until later in the protocol) is prepared. The OPD substrate solution contains 0.1% Triton X100 (1 ml of Triton X100 diluted with 99 ml 55 mM Tris (pH 8.0); diluted 1:10 in substrate solution), 1 mM OPD, 1 mM H₂O₂ (made fresh and diluted in 55 mM Tris and substrate solution), and enough 55 mM Tris (pH 8.0) to bring to final volume needed (typically 10 ml).

When adhesion time is up, the plate and control stock eosinophils are removed from incubator. The wash plate is washed 3× with TBS, pH 8.0. 100 μl of HBSS/0.2% BSA is added to each reaction well, and 100 μl of 100% control stock eosinophils is added to 3 additional wells. OPD (50 mM OPD can be produced by weighing approximately 100 mg o-phenylenediamine diHCl and dispersing about 0.2 ml/eppendorf and freezing at −80° C.) is added to substrate solution and mixed. 100 μl of substrate solution is added to all wells and incubated 30 minutes at room temperature or long enough for color to develop. The reaction is stopped by adding 50 μl of 4 M H₂SO₄.

The plate is read immediately on a 96-well ELISA spectrophotometer at 490 nm. If necessary, the plate can be held for up to 60 minutes in dark before reading. The percent eosinophil adherence is calculated by dividing the OD₄₉₀ for experimental wells into the OD₄₉₀ for the 100% control wells and multiplying by 100.

The occurrence of the amount of activation of β₁ integrin above a minimum threshold in conjunction with detecting P-selectin associated with eosinophils can be used as an indicator of a decreased lung function. The occurrence of the amount of activation of β₁ integrin below a minimum threshold in conjunction with detecting P-selectin associated with eosinophils can be used as an indicator of an increased lung function. The level of β₁ integrin activation correlates inversely with lung function, which can be measured by, for example, FEV₁. The level of β₁ integrin activation in conjunction with detecting P-selectin associated with eosinophils is a predictor of lung function and asthma control.

Quantifying the Total Number of Eosinophils in the Sample:

The total number of eosinophils present in a sample can be quantified. This accounts for non-eosinophils that are positive for P-selectin, such as monocytes and neutrophils. The total number of eosinophils in a sample can be quantified using e.g., a flow cytometer and an antibody, such as anti-CD14 or CD16, which distinguishes eosinophils from other cells present in the sample.

In using an immunoassay that captures P-selectin-positive cells, the total number of captured P-selectin-positive eosinophils (eos) in the sample can be quantified using an eosinophil-specific assay, such as EPO assay described above, using an adhesion protein ligand that binds eosinophils, such as VCAM-1 in place of the P-selectin antibody. An EPO assay is routinely used to measure adhesion of eosinophils in cell-biological experiments and can be used to detect attached eosinophils even in whole blood or other mixed population of leukocytes (i.e., the assay will determine the number of attached eos in the presence of other cells, e.g., other attached leukocytes). Measuring the attachment of eosinophils to an immobilized P-selectin antibody with the EPO assay is similar in principle to the EPO assay routinely used to measure eosinophil adhesion to adhesive protein ligands such as VCAM-1. (Sedgwick et al.)

The EPO adhesion assay can be used with at least about 40 μl of blood because the assay can be used to detect about 500 eos or fewer (compared to the background). The sensitivity can be increased by extending the enzyme assay longer than the standard time period. For example, to attach 500 eos in a sample obtained from a person with a low-end number of eos and a low-end N29 reactivity, such as 25% N29-binding eos, 40 μl of blood is required (giving 2000 eos, of which a quarter would attach to N29). If the enzyme assay is enhanced, then even less blood can be used.

Other methods of quantifying the total number of eosinophils in the sample can also be used as are known and used in the art. For example, the dye eosin can be used because eosinophils take up eosin.

The analysis of a sample for P-selectin associated eosinophils in combination with quantifying the total number of eosinophils provides a method of determining the percent of P-selectin positive eosinophils in a sample.

Assay to Identify Agents to Disrupt P-Selectin Mediated Eosinophil β-1 Integrin Activation

P-selectin mediated eosinophil β-1 integrin activation is a therapeutic target for the treatment of asthma or other biological condition, for example a respiratory disorder. Agents that inhibit or impede P-selectin-mediated eosinophil β-1 integrin activation would be useful in the treatment of asthma.

Another embodiment of the invention is a method for identifying an agent that affects P-selectin-mediated eosinophil β-1 integrin activation. Examples of such methods involve contacting the agent to a composition comprising eosinophils and P-selectin under conditions sufficient to allow interaction between the cell and the agent. The amount of β-1 integrin activation in the cell contacted with the agent can be evaluated and compared to the amount of β-1 integrin activation in a control cell not treated with the agent. A statistically significant difference in the amount of β-1 integrin activation in the cell contacted with the agent, as compared to the control cell not treated with the agent, identifies the agent as one that affects the ability P-selectin to mediate β-1 integrin activation.

For example, whole blood can be drawn into green-top heparin tubes (Vacutainer Systems), or purified blood eosinophils can be resuspended at 5×10⁶/ml in Roswell Park Memorial Institute (RMPI) 1640 medium (Mediatech, Herndon, Va.) with 0.2% bovine serum albumin (BSA), from volunteer donors and split into two samples. The first sample will be incubated with recombinant soluble extracellular domain of P-selectin (R&D Systems, Minneapolis, Minn.) and incubated for 1 h at 37° C. The second sample will be incubated for 1 h at 37° C. with recombinant soluble extracellular domain of P-selectin and an agent. The ability of the agent to inhibit or impede P-selectin mediated eosinophil β-1 integrin activation will be tested using the VCAM-1 adhesion assay.

Briefly, wells in six-well culture plates (Corning, Corning, N.Y.) will be coated with 1 ml of different concentrations of recombinant soluble extracellular 7-domain-VCAM-1 in Tris-buffered saline (TBS) for 2 h at 37° C. (Johansson M W, et al. 2004) The VCAM-1-coated wells will be washed with TBS, blocked with 0.1% gelatin (for eosinophils) in TBS for 30 minutes at 37° C., and washed with TBS. One ml of each sample (with and without agent) in RPMI 1640 with 0.2% BSA will be plated in each well. After incubation for 1 h at 37° C., non-adherent cells will be harvested, spun down for 10 minutes at 4° C. at 1,200 revolutions per minute (rpm), resuspended in 400 μl FACS buffer (phosphate-buffered saline [PBS] with 2% BSA and 0.2% NaN₃), and processed for flow cytometry. The flow cytometry procedure will be as described in Example 2 infra. The proportion of adherent cells, which is indicative of β-1 integrin activation, will be compared between the sample without agent and the sample with agent. An agent that produces a substantial reduction in the proportion of adherent cells may be functioning by inhibiting or impeding P-selectin-mediated eosinophil β-1 activation. These agents could be used in the treatment of asthma.

Kits to Detect P-Selectin:

The invention further provides kits for determining the amount of P-selectin associated with eosinophils. The kits also can be used for correlating the level of P-selectin associated with circulating eosinophils and β-1 integrin activation state.

In one embodiment of the kit, the kit includes a positive control including but not limited to soluble P-selectin and cells that express P-selectin. The kit can also include a negative control including cells that lack P-selectin. The kit can also include a binding partner of P-selectin, such as an antibody that binds to P-selectin. An example of an antibody that can be used in the kit is AC1.2.

The positive control can be platelets, either activated platelets or non-activated. If the platelets are non-activated, thrombogenic agents such as histamine and thrombin can also be included in the kits. Prior to using the platelets, they would be activated upon addition of the thrombogenic agent. The negative control can be any cell or cell line that does not express P-selectin.

The kit also can include a binding partner of activated β₁ integrin, such as an anti-β₁ integrin monoclonal antibody that binds to β₁ integrin when β₁ integrin is activated. An example of antibodies that can be used in the kit includes but is not limited to mAb N-29, mAb 8E3, mAb HUTS-21, mAb 9EG7, and mAb 12G10.

A secondary antibody that binds to the first antibody or to the complex of the first antibody and its antigen can be included in the kit. The secondary antibody can include a detectable label. A development reagent, such as a substrate for an antibody-linked enzyme can also be included where the secondary antibody has an enzyme conjugated to it.

The kit can be employed in laboratory settings and outside the laboratory. The kit can be adapted to be portable and for use in a patient's home. For example, the kit can be adapted to be in a format like that of home pregnancy test kits in which antibody embedded filter paper is included for use in contacting a sample from the patient. The resultant binding of the antibody with the sample could then produce a positive or negative result, or could generate a graduated result that would be compared to a representation of known results.

The kit may also include instructions. The instructions may provide information on incubation times, dilution factors for various antibodies, appropriate secondary antibodies, and general information on how to perform the assays.

### EXAMPLES

The following Examples are provided for illustrative purposes only. The Examples are included herein solely to aid in a more complete understanding of the presently described invention. The Examples do not limit the scope of the invention described or claimed herein in any fashion.

### Example 1

We investigated the correlation between β₁ integrin activation state and the level of P-selectin associated with eosinophils in whole blood. Participants were part of the Virus Induced Asthma Exacerbation (VIAX) study and consisted of mild/moderate asthmatic subjects studied during the acute and resolution phases of an upper respiratory tract viral infection. β₁ integrin activation state was assessed with activation-sensitive anti-β₁ mAb N29. In addition, other monoclonal antibodies for β1 were used to test whether the correlation with P-selectin was specific to a specific conformational state of β1 integrin.

Methods

1. Subjects

Twenty-three subjects with mild or moderate asthma enrolled in the VIAX study were studied during the acute and resolution phases of an upper respiratory tract viral infection. Subjects were recruited and screened, then asked to return at the onset of an upper respiratory tract viral infection. Subjects had eleven visits: the first four during the acute phase, visits No. 5-7 during the recovery phase until symptoms of the viral infection were gone, and visits No. 8-11 during the resolution phase, which was typically six to twelve weeks after the acute phase. All subjects had a positive skin prick test to at least one of twelve aeroallergens; did not have a history of severe episodes of asthma with respiratory infections; did not have an ICS dose of >400 μg fluticasone/day or equivalent, or Advair >250/50; did not have an albuterol use of ≧6 puffs/day; were non-smokers; and were not pregnant or breast-feeding. All studies were approved by the University of Wisconsin-Madison Health Sciences Human Subjects Committee. Informed consent was obtained from each subject before participation.

2. Assessments

Clinical assessments were performed as previously described. (Johansson M W, et al. 2006). Spirometry for the determination of FEV₁ was carried out according to American Thoracic Society standards (Am J Respir Crit Care Med 1995; 152:1107-36) at all visits of the VIAX study, except at visits No. 2 and 9. Methacholine challenge to assess airway responsiveness was performed at visit No. 8 as previously described. (Crapo R O, et al.) Blood was drawn at visits No. 3 and 11 into standard lavender-top EDTA tubes (BD Vacutainer Systems, Franklin Lake, N.J.) and directly processed for flow cytometry at room temperature as described below.

3. Antibodies

Activation-sensitive anti-β₁ integrin mAbs 8E3 (Coe A P, et al.) and 12G10 (Mould A P, et al., 2005) were obtained from Martin Humphries (Wellcome Trust Center for Extracellular Matrix Research, Manchester, UK). Anti-β₁ MAR4; anti-β₂ L130, activation-sensitive anti-β₁ mAbs HUTS-21 (Luque A et al.) and 9EG7 (Bazzoni G, et al); phycoerythrin (PE)-conjugated goat anti-mouse and anti-rat immunoglobulin (Ig) G; anti-P-selectin AC1.2; anti-PSGL-1 KPL-1; FITC-conjugated anti-CD14 and anti-CD16; allophycocyanin (APC)-conjugated anti-P-selectin AK-4; and isotype controls mouse IgG₁, κ (clone A112-2), mouse IgG₂ₐ, κ (G155-178), and rat IgG₂ₐ, κ (A110-2) were from BD Biosciences. Activation-sensitive anti-β₁ N29 (Wilkins J A, et al.) and FITC-conjugated goat anti-mouse IgG were from Chemicon (Temecula, Calif.).

4. Flow Cytometry

Flow cytometry was performed essentially as described by Johansson et al. 2008). Whole blood (100 μl) was incubated with 0.5 μg primary antibody or isotype control in 100 ml FACS buffer for 30 minutes. After primary antibody incubation, samples were washed with 1 ml PBS, washed with 250 μl FACS buffer, and resuspended in 250 μl FACS buffer containing PE-conjugated secondary antibody (2 μg/ml). After incubation for 30 minutes, samples were washed again with PBS, resuspended in 100 μl FACS buffer with FITC-conjugated anti-CD14 (0.125 μg) and anti-CD16 (0.625 μg) and incubated for 30 minutes. Red blood cells were lysed by incubation with 2 ml FACS lysing solution (BD Biosciences) for 10 minutes, followed by centrifugation. Incubations were at room temperature until after red blood cell lysis and then at 4° C. Samples were washed with 500 FACS buffer, resuspended in 250 μl FACS fixative (1% paraformaldehyde, 67.5 mM sodium cacodylate, 113 mM NaCl, pH 7.2), stored at 4° C. in the dark, and washed with 1 ml PBS and resuspended in 250 μl FACS buffer just prior to data collection. Fixation did not decrease signals (not shown). In a variant of this protocol, blood was incubated with primary and PE-conjugated secondary antibodies as above, followed by resuspension in 100 μl FACS buffer with APC-conjugated anti-P-selectin (20 μl) and incubation for 30 minutes. Red blood cells were lysed and samples were fixed, stored, and prepared for data collection as above.

Data were collected from 30,000-170,000 events with whole blood samples or 10,000 purified eosinophils or EoL-3 cells, using a FACS Calibur (BD Biosciences; available through the Flow Cytometry Facility, Comprehensive Cancer Center, University of Wisconsin-Madison). Mid-range one-peak “rainbow” fluorescent beads (catalog No. RFP-30-5A, lot No. W02; Spherotech, Libertyville, Ill.) were first run at setup to check the optical alignment and set the sensitivity of the different detectors at a standardized fluorescence intensity, thus optimizing comparisons of data among subjects. Data were collected using Cellquest (BD Biosciences) and analyzed using FlowJo (TreeStar, Ashland, Oreg.). Post-data collection compensation for possible overlap between fluorochromes was performed using matrix algebra by FlowJo to remove any degree of subjectivity introduced by the manual compensation performed at setup. Eosinophils in whole blood were gated based both on scattering and lack of staining with anti-CD14 and anti-CD16, i.e., the cells that were analyzed for PE signal fit two criteria for eosinophils by being gated inside both characteristic regions in a plot of side scatter versus FITC staining and a plot of side versus forward scatter. Similarly, neutrophils and monocytes were gated based on scattering and FITC staining for CD16 and CD14, respectively. In the variant protocol with APC-anti-P-selectin staining, leukocytes were gated based on side and forward scatter. Data are expressed as specific geometric mean channel fluorescence (gMCF; specific gMCF=gMCF with a specific integrin mAb-gMCF with isotype control).

5. Statistics

The Spearman rank correlation test was used to analyze correlations. For subjects from which data were available from more than one visit, data from only one (selected by coin toss) of those visits were included in the correlation analyses. A level of probability (P)≦0.05 was considered significant. Analyses were performed using Prism 3.0 (GraphPad, San Diego, Calif.).

Results and Discussion

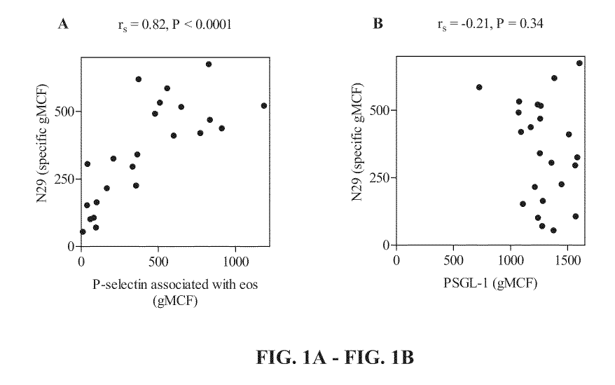

The demographics for the study participants are provided in Table 1. There was a striking correlation between β₁ integrin activation state, as assessed with activation-sensitive anti-β₁ mAb N29, on eosinophils and the level of P-selectin associated with eosinophils in whole blood (FIG. 1A; Table 2). Eosinophil N29 reactivity did not correlate with eosinophil PSGL-1 expression (FIG. 1B).

TABLE 1
Characteristics of mild/moderate asthmatic subjects studied
during and after an upper respiratory tract viral infection

| Subject | Age | | PC₂₀ | FEV₁ | |
|---|---|---|---|---|---|
| (No.)[ID] | (years) | Sex | (mg/ml) | (L) | (% pred.) |
| 1 [4] | 20 | F | 0.5 | 3.8 | 101 |
| 2 [6] | 21 | F | ? | 2.5 | 84 |
| 3 [7] | 27 | F | 1.0 | 2.8 | 74 |
| 4 [8] | 32 | M | 3.7 | 3.6 | 81 |
| 5 [11] | 22 | M | 12 | 5.4 | 107 |
| 6 [14] | 20 | F | 4.6 | 3.2 | 113 |
| 7 [15] | 20 | F | 1.4 | 3.0 | 82 |
| 8 [16] | 22 | F | ? | 4.1 | 84 |
| 9 [18] | 25 | M | 0.2 | 4.1 | 84 |
| 10 [19] | 28 | M | 25 | 5.2 | 125 |
| 11 [20] | 28 | F | 25 | 3.5 | 110 |
| 12 [21] | 21 | F | 25 | 3.9 | 93 |
| 13 [23] | 37 | F | 25 | 2.4 | 79 |
| 14 [26] | 23 | F | 25 | 3.5 | 94 |
| 15 [28] | 36 | F | 1.2 | 3.2 | 100 |
| 16 [29] | 28 | M | ? | 3.9 | 88 |
| 17 [32] | 44 | F | 8.3 | 2.5 | 88 |
| 18 [34] | 42 | F | 12 | 2.9 | 96 |
| 19 [36] | 19 | F | ? | 2.3 | 74 |
| 20 [38] | 18 | M | 0.4 | 4.4 | 93 |
| 21 [39] | 20 | F | ? | 4.0 | 111 |
| 22 [41] | 18 | M | 25 | 3.8 | 101 |
| 23 [42] | 27 | M | ? | 3.4 | 93 |

F, female; FEV₁, forced expiratory volume in 1 s; M, male; PC₂₀, provocative concentration of methacholine producing a 20% fall in FEV₁; % pred, % of predicted. PC₂₀ values are from visit 8 and FEV₁ values are from visit 10, both during the resolution phase (exceptions: FEV₁ value for subject 2 is from visit 5; for subject 8 from visit 4; and for subjects 16, 19, 21, 21, and 23 from visit 7). Medians with 25^{th} and 75^{th} percentiles: Age 23 years (20, 30); PC₂₀ 8.3 mg/ml (1.1, 25): FEV₁ 3.5 L (3.0, 4.0), 93% pred. (84, 108).

TABLE 2
Correlations between N29 epitope expression and associated P-
selectin on eosinophils, neutrophils, or monocytes (n = 23)

| Cell type | rₛ | P |
|---|---|---|
| Eosinophils | 0.82 | <0.0001 |
| Neutrophils | 0.81 | <0.0001 |
| Monocytes | 0.48 | 0.02 |

n, number of subjects; P, probability; rₛ, Spearman rank correlation coefficient.

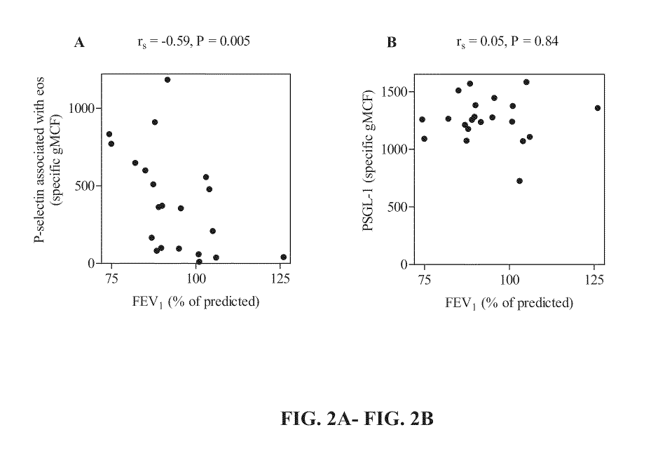

There was a trend to a lower FEV₁ in subjects during the acute phase of the infection (median 91% of predicted, 25^{th} and 75^{th} percentiles 84 and 102) than during the resolution phase (median 95% of predicted, 25^{th} and 75^{th} percentiles 88 and 102), but this difference did not reach significance (P=0.54, Mann-Whitney U test; P=0.31, Wilcoxon signed-rank test). However, eosinophil-associated P-selectin in a blood sample correlated inversely with FEV₁ measured at the same visit (FIG. 2A; Table 3), whereas eosinophil PSGL-1 did not (FIG. 2B). Thus, the amount of eosinophil-bound P-selectin, but not the amount of its counter-receptor, PSGL-1, correlated with β₁ activation state and decreased lung function.

TABLE 3
Correlations between associated P-selectin on eosinophils,
monocytes, or neutrophils in whole blood from mild/moderate
asthmatic subjects (n = 21) and FEV₁ during and
after an upper respiratory tract infection.

| Cell type | rₛ | P |
|---|---|---|
| Eosinophils | −0.59 | 0.005 |
| Neutrophils | −0.48 | 0.03 |
| Monocytes | −0.38 | 0.09 |

FEV₁, forced expiratory volume in 1 s (expressed as % of predicted); n, number of subjects; P, probability; rₛ, Spearman rank correlation coefficient.

Correlation Between P-Selectin and Specific Conformational States of β-1 Integrin

In order to examine how specific the correlations was for eosinophils, we also gated and analyzed neutrophils and monocytes in the same blood samples. Neutrophil N29 reactivity and associated P-selectin correlated in a manner similar to that of eosinophils, whereas with monocytes these signals correlated to a much lower degree (Table 2). Neutrophil-associated P-selectin correlated with FEV₁ to a lower degree than did eosinophil-associated P-selectin and monocyte-associated P-selectin did not correlate with FEV₁ (Table 3). Therefore, as with eosinophils, neutrophil-bound P-selectin correlated with neutrophil β₁ activation state.

To test whether the correlation with P-selectin was specific for β₁ conformations that exposed the N29 epitope or more generally reflected activated conformations of β₁, a panel of other activation-sensitive β₁ mAbs were studied in a subset of the subjects (11 subjects). There was variable relatively high eosinophil expression of the epitope for mAb 12G10; low to variable expression of the 8E3 and HUTS-21 epitopes; and no, low, or variable expression of the 9EG7 epitope (data not shown). In this subset of subjects, for which the correlation between eosinophil N29 reactivity and associated P-selectin was similar to that with all subjects, there were similar correlations between eosinophil expression of the other β₁ activation epitopes and P-selectin (Table 4). Further, the expression of these epitopes correlated with N29 epitope expression, with 8E3 most closely tracking N29, followed by 12G10 and HUTS-21, whereas 9EG7 tracked N29 to a lower degree (Table 5).

TABLE 4
Correlations between expression of epitopes for activation-sensitive
β₁ integrin mAbs and associated P-selectin on eosinophils in whole
blood from a subset of the mild/moderate asthmatic subjects (n =
11) during and after an upper respiratory tract viral infection.

| β₁ integrin mAb | rₛ | P |
|---|---|---|
| N29 | 0.85 | 0.002 |
| 8E3 | 0.88 | 0.0007 |
| 12G10 | 0.89 | 0.0005 |
| HUTS-21 | 0.82 | 0.003 |
| 9EG7 | 0.83 | 0.003 |

mAb, monoclonal antibody; n, number of subjects; P, probability; rₛ, Spearman rank correlation coefficient.

TABLE 5
Correlations between N29 epitope expression and expression of epitopes
for other activation-sensitive β₁ integrin mAbs in whole blood
from a subset of the mild/moderate asthmatic subjects (n =
11) during and after an upper respiratory tract viral infection.

| β₁ integrin mAb | rₛ | P |
|---|---|---|
| 8E3 | 0.96 | <0.0001 |
| 12G10 | 0.91 | 0.0003 |
| HUTS-21 | 0.89 | 0.0005 |
| 9EG7 | 0.61 | 0.05 |

mAb, monoclonal antibody; n, number of subjects; P, probability; rₛ, Spearman rank correlation coefficient.

Correlation Between β₁ Integrin Activation State and P-Selectin Associated with Eosinophils in Individual Eosinophils

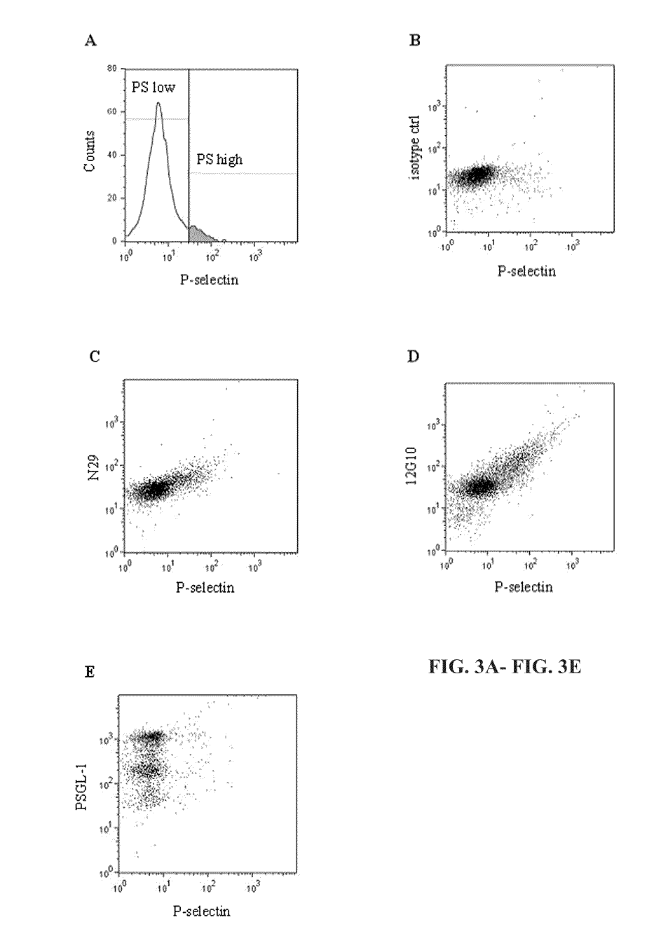

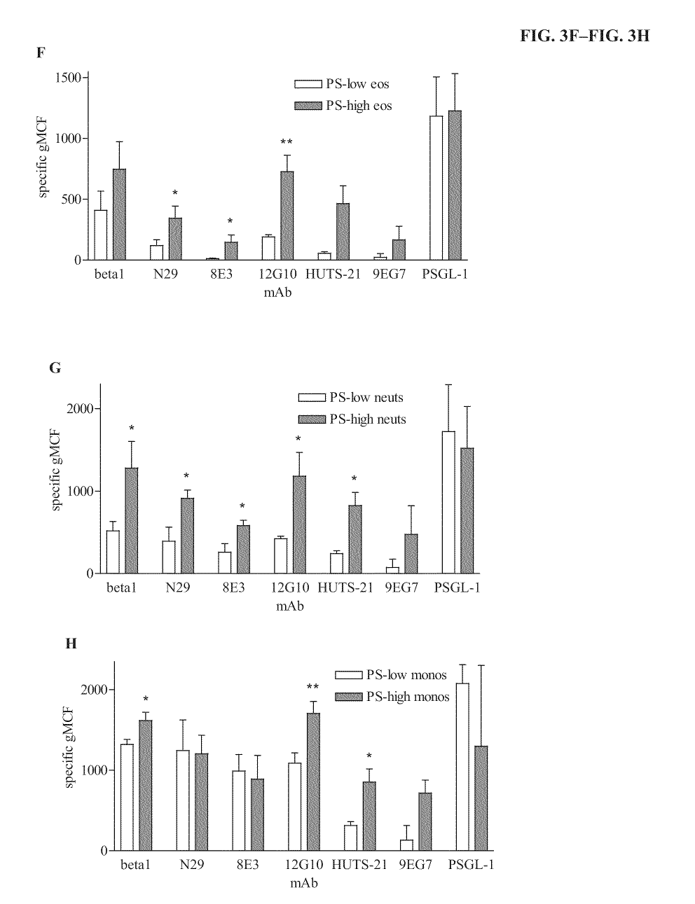

To investigate the association of P-selectin and β₁ activation state at the level of individual eosinophils, a dual labeling protocol was designed using APC-labeled anti-P-selectin combined with different primary mAbs, including mAbs for the β₁ activation epitopes and PSGL-1, and then detected by PE-conjugated secondary antibody. FIG. 3A-FIG. 3E reports the results from one representative VIAX study subject. Two populations of eosinophils were identified: one with low level of associated P-selectin (as depicted by white region in FIG. 3A) and one with high (as depicted by shaded region in FIG. 3A) level of associated P-selectin (comprising 91% and 9%, respectively of the eosinophils in this example). The P-selectin-low and P-selectin-high populations had similar levels of background fluorescence with mouse IgG₁+PE-conjugated secondary antibody (FIG. 3B). In contrast, staining with activation-sensitive β₁ mAbs followed by PE-labeled secondary antibody revealed that the P-selectin-high population showed a “tail” of higher β₁ activation mAb reactivity. The results from staining with activation-sensitive β₁ mAb N29 is shown in FIG. 3C and the results from staining with β₁ mAb 12G10 are shown in FIG. 3D. β₁ mAb 8E3 and 9EG7 were also tested but this data is not shown. There was no difference between P-selectin-low and P-selectin-high populations with respect to PSGL-1 expression (FIG. 3E). FIG. 3F summarizes the specific signals in the P-selectin-low and P-selectin-high eosinophil populations in three subjects after subtraction of fluorescence with isotype control. Eosinophil expression of β₁ activation epitopes N29, 8E3, HUTS-21, and 9EG7 was low or very low in the P-selectin-low population. N29, 8E3, and 12G10 epitope expression was significantly higher in the P-selectin-high eosinophil population as compared to the P-selectin-low population. There were trends to higher epitope expression for β₁ mAB HUTS-21 and 9EG7. There was no detectable difference in PSGL-1 expression between the two populations. These results indicate that eosinophil expression of β₁ activation epitopes correlates with eosinophil-associated P-selectin within one individual.

Neutrophils and monocytes similarly were divided into two populations of P-selectin. In the P-selectin-low populations of these leukocyte types, only 9EG7 had a very low expression (FIG. 3G and FIG. 3H). With neutrophils, N29, 8E3, 12G10, and HUTS-21 epitopes had significantly higher expression in the P-selectin-high population (FIG. 3G). With monocytes, 12G10 and HUTS-21 epitopes had significantly higher expression in the P-selectin-high-population; whereas there was no difference in expression of the N29 and 8E3 epitopes in the P-selectin high and P-selectin low populations. Thus, with monocytes, in contrast to eosinophils and neutrophils, P-selectin-low cells appear to have a β₁ activation state high enough to maximally expose N29 and 8E3 epitopes. PSGL-1 expression on P-selectin-high monocytes was very variable among the subjects and was lower than on the P-selectin-low population in two subjects, indicating that, in at least some subjects, a high level of bound P-selectin may be associated with PSGL-1 downregulation or shedding on monocytes.

These data reveal a correlation between the level of P-selectin associated with eosinophils and the activation state of β₁ integrins, as assessed with the activation-sensitive mAb N29, on circulating eosinophils in samples from mild/moderate asthmatic subjects during and after an upper respiratory tract viral infection. These results support the hypothesis that an interaction between P-selectin and eosinophils is responsible for an enhanced β₁ activation state. β₁ activation, therefore, likely can be considered as reporting the degree of lung endothelial cell and/or platelet activation in mild/moderate asthmatics. Eosinophil signals for both P-selectin and N29 varied greatly among subjects from very low to relatively high. In contrast, eosinophil expression of PSGL-1, the counter-receptor for P-selectin, was relatively high in all subjects, was less variable, and did not correlate with N29 (nor with the P-selectin signal, data not shown). Thus, the amount of PSGL-1 does not seem to influence eosinophil β₁ activation state.

There was a trend to decreased FEV₁ during the acute phase of the infection, but FEV₁ varied considerably among subjects. The strength of the eosinophil P-selectin signal, but not the PSGL-1 signal, correlated inversely with FEV₁, supporting a scenario that P-selectin association with eosinophils favors enhanced eosinophil arrest, extravasation, and recruitment to the airway, and indicating that eosinophil-associated P-selectin may be a novel potential marker in mild/moderate asthma.

A similar correlation between N29 and P-selectin signals was found on neutrophils in the same samples, and to a much lower degree on monocytes. Neutrophil-associated P-selectin correlated less well with FEV₁ than did eosinophil-associated P-selectin, and monocyte-associated P-selectin did not correlate with FEV₁. Overall, the results are compatible with the idea that the eosinophil is the leukocyte type whose activation and egress is important in mild/moderate asthma.

Results with other activation-sensitive β₁ mAbs, in addition to N29, tested on a subset of the asthmatic subjects, taking into consideration the mapping of these mAbs onto integrin structures (Luo B H, et al.; Humphries M J, et al., 2004; Mould A P, et al., 2005; Humphries M J, et al., 2003) demonstrate that eosinophil β₁ conformation varied greatly. In some subjects, there was very low N29 reactivity, which is likely representative of β₁ being mostly in the bent, unextended conformation. In other subjects, β1 clearly was reacting with all the mAbs and likely being, at least partly, in the extended and “swung-out” high-affinity conformation with the α and β integrin subunit legs separated, and having an activated ligand-binding domain. Eosinophil reactivity with all the β₁ activation mAbs, like that with N29, correlated with the level of eosinophil-associated P-selectin. Further, reactivity with the other mAbs correlated with that of N29. Taken together, these result indicate that P-selectin binding to eosinophils is associated with a spectrum of coordinated conformational changes in β₁ that involves, in addition to unbending/extension and exposure of the N29/8E3 epitopes in the PSI domain, activation of the ligand-binding I domain activation, “swing-out” of the hybrid domain, and separation of the α and β subunit legs; although the degree to which these coordinated events occur varies among subjects.

Within one individual, P-selectin binding also appears to be associated with eosinophil β₁ conformational changes. Thus, it seems that those eosinophils with no or little bound P-selectin have no-intermediate affinity β₁ conformations, with some, relatively low, degree of unbending and ligand domain activation but with neither “swing-out” nor leg separation. Those eosinophils with a high level of bound P-selectin, on the other hand, have conformations with all epitopes more exposed, including the hybrid domain having “swung-out” and α and β subunit legs separated. The situation with neutrophils was reminiscent of that with eosinophils, although baseline mAb reactivity appeared somewhat higher than on eosinophils, whereas the situation with monocytes was different. β₁ on all monocytes within one individual seem to be in unbent, extended conformations with the N29/8E3 epitopes in the PSI domain fully exposed, regardless of the level of bound P-selectin. However, a high level of bound P-selectin was associated with further I domain activation and hybrid domain “swing-out.” Overall, P-selectin binding appears associated with the relatively greatest β₁ conformational changes on eosinophils, somewhat similar on neutrophils, and a more limited set of changes on monocytes. In contrast to the β₁ activation epitopes, the expression of the counter-receptor PSGL-1 of an individual leukocyte appears independent of the level of bound P-selectin, consistent with the relatively constant PSGL-1 expression seen when comparing across subjects.

### Example 2

Experiments were performed to determine whether addition of soluble recombinant extracellular domain of P-selectin triggers enhanced β₁ integrin activation state on eosinophils in whole blood or purified eosinophils in vitro. The β₁ mAb N29 was used to assess the activation-state of β₁ with and without the addition of P-selectin.

Methods

1. Cells

Eosinophils were purified from peripheral heparinized blood of normal, allergic rhinitic, or allergic asthmatic volunteers by negative selection using a cocktail of anti-CD16, anti-CD14, and anti-CD3 magnetic beads in the AutoMACS system (Miltenyi, Auburn, Calif.). (Hansel T T, et al.) The source of the antibodies is as described in the Materials and Methods in Example 1 supra. The purity of eosinophils was >99% as determined by Diff-Quik staining (Dade, Düdingen, Switzerland). Viability was >98% as assessed by staining with propidium iodide and annexin V-fluorescein isothiocyanate (FITC) (BD Biosciences, San Diego, Calif.).

2. Addition of Soluble P-Selectin

Whole blood drawn into green-top heparin tubes (Vacutainer Systems), or purified blood eosinophils resuspended at 5×10⁶/ml in Roswell Park Memorial Institute (RMPI) 1640 medium (Mediatech, Herndon, Va.) with 0.2% bovine serum albumin (BSA), from volunteer donors were pre-incubated with or without different concentrations of added recombinant soluble extracellular domain of P-selectin (R&D Systems, Minneapolis, Minn.) for 1 h at 37° C. before being processed for flow cytometry as below.

3. Flow Cytometry

Purified eosinophils were analyzed essentially as described (Johansson M W, et al., 2004) All steps were at 4° C. One hundred μl eosinophils (5×10⁵) suspended in FACS buffer were incubated with 0.5 μg primary antibody or isotype control in 100 μl FACS buffer for 30 minutes, washed with 250 μl FACS buffer, resuspended in 250 μl FACS buffer containing PE-conjugated secondary antibody (2 μg/ml), and incubated for 30 minutes. Cells were fixed, stored, and prepared for data collection as described in the Flow cytometry section of the Material and Methods section of Example 1 supra. Data collection was as described in Material and Methods section of Example 1 supra. Flow cytometry of whole blood was performed as described above.

4. Statistics

The means of populations were compared using two-tailed Student's t test. A level of probability (P)≦0.05 was considered significant. Analyses were performed using Prism 3.0 (GraphPad, San Diego, Calif.).

Results and Discussion

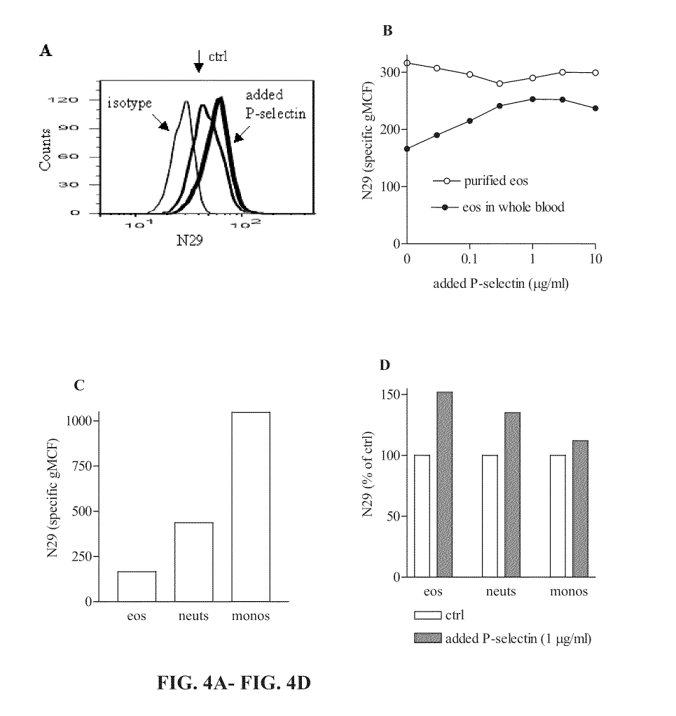

Experiments were performed to address whether addition of soluble recombinant extracellular domain of P-selectin triggers enhanced β₁ integrin activation state on eosinophils in whole blood or purified eosinophils in vitro. N29 epitope expression was higher on purified eosinophils, on the average 1.4-fold higher, as compared to eosinophils in whole blood (n=5 donors from whom paired samples of blood and purified eosinophils were obtained) (FIG. 4B). N29 reactivity increased in whole blood with a 1 h pre-incubation with added P-selectin (FIG. 4A and FIG. 4B), but not on purified eosinophils (FIG. 4B). An increase in N29 reactivity in blood in response to P-selectin was observed in four of five donors from whom paired samples of blood and purified eosinophils were obtained. With regard to the purified eosinophils, P-selectin did not increase N29 reactivity in any of the samples.

Dose responses in blood samples from different donors were variable; maximal N29 epitope expression was achieved at concentrations of added P-selectin of 0.1-1 μg/ml (FIG. 4B) depending of the donor. Of seven donors for whom studies were done on whole blood, six showed an increase in N29 epitope reactivity in response to pre-incubation with P-selectin, up to 1.6-fold, compared to control pre-incubation (P=0.009, paired t test). These results demonstrate that P-selectin is capable of causing enhanced β₁ activation state on eosinophils in blood.

In the absence of added P-selectin, N29 reactivity of the different leukocytes was in the following order: eosinophils displayed the weakest reactivity; neutrophils displayed greater reactivity than eosinophils but less reactivity than monocytes, and monocytes displayed the strongest reactivity (FIG. 4C). The relative increase in N29 reactivity in response to added P-selectin in the same donor was in the opposite order: eosinophils displayed the greatest relative increase in reactivity; neutrophils displayed a greater relative increase in reactivity compared to monocytes but less than eosinophils and monocytes displayed the smallest relative increase in reactivity (FIG. 4D). In addition, the effect of P-selectin on eosinophils in blood in vitro was specific for β₁ integrins, since P-selectin did not increase the expression of the epitope for mAb24, which is an activation-sensitive antibody against β₂ integrins (data not shown). (Dransfield I, et al.) (Lu C, et al.) Conversely, mAb24 reactivity on eosinophils in blood was increased by IL-5, in accordance with the literature that IL-5 activates α_{M}β₂ on purified eosinophils (Barthel S R, et al.) (Zhu X et al.) but there was no detectable activation by P-selectin (data not shown).

Results of in vitro experiments were compatible with the analyses of samples from the mild/moderate asthmatics. Baseline eosinophil β₁ activation state was enhanced by the addition of exogenous soluble P-selectin. Baseline state was higher on neutrophils and even higher on monocytes, and P-selectin enhanced it relatively less on neutrophils than on eosinophils and even less on monocytes. The dose response of added P-selectin varied among donors, not surprisingly considering the variation in baseline level, and maximal effect on eosinophil N29 epitope expression was obtained at a final concentration of added P-selectin of 0.1-1 μg/ml. This is 10-100-fold less than the concentration used in the literature to observe an effect on HUTS-21 reactivity on monocytes. (da Costa et al.) The concentration of soluble P-selectin in plasma of normal donors is between 20-40 ng/ml. Thus, it appears that an addition of 100 ng/ml corresponding to only a four-six-fold increase can cause an enhanced activation state of eosinophil β₁.

The plasma P-selectin concentration in disease states; such as hemolytic uremic syndrome, thrombotic thrombocytopenic purpura, and acute myocardial infarction; have been reported to be up to about three-fold that of normal donors, so the concentrations of added P-selectin in our experiments should be within a realistic in vivo range. (Gearing A J, et al.; Ikeda H, et al.; Xu Dy, et al.) How high plasma P-selectin levels in asthmatics may reach appears uncertain, but likely at least four-fold that of normal donors. Plasma was not collected from the subjects in this study. Concentrations of about 70 ng/ml were obtained in asthmatics with a dual response phenotype after whole lung allergen challenge, (Kowal K, et al.) and we have measured about 70 ng/ml in samples from asthmatics in the SARP study (not shown). Likely, higher levels would be reached locally and/or during an exacerbation. In the literature, soluble P-selectin in asthmatics has, unfortunately, often been measured in serum (Sjosward K N, et al.; Oymar K, et al.); the absolute concentrations obtained in such analyses are, however, of dubious value because of the unknown contribution from platelet activation during clotting of whole blood. It is possible that activated platelets expressing surface P-selectin may exert a stronger effect than soluble P-selectin, due to expected higher avidity if P-selectin is presented to the eosinophils on a surface. Further, the source(s) of the P-selectin found associated with eosinophils in blood remains to be determined. It may be bound from plasma and/or be on the surface of activated platelets associated with the eosinophils, and/or perhaps somehow be directly derived from the surface of activated endothelium. We have observed, by immunofluorescence microscopy of cytospins of purified eosinophils, that a proportion of eosinophils have dot- or rod-like structures, one to a few nm long, positive for the platelet proteins, α_{Iib} integrin subunit and thrombospondin-1 (not shown), consistent with the structures being eosinophil-associated platelets or platelet fragments. Thus, at least some of the eosinophil-bound P-selectin may be due to this “satellitism” of activated platelets or platelet fragments.

### Example 3

The correlation between the activation state of β₁ and the affinity for β₁ integrins for ligand binding was investigated. An adhesion assay was developed to monitor binding to VCAM-1 coated wells and the activation state of β₁. Eosinophils purified from whole blood and human EoL-3 leukemic eosinophilic cells were analyzed.

Methods

1. Cells

Eosinophils were purified from peripheral heparinized blood of normal, allergic rhinitic, or allergic asthmatic volunteers by negative selection using a cocktail of anti-CD16, anti-CD14, and anti-CD3 magnetic beads in the AutoMACS system (Miltenyi, Auburn, Calif.). (Hansel T T, et al.) The source of the antibodies is as described in the Materials and Methods in Example 1 supra. The purity of eosinophils was >99% as determined by Diff-Quik staining (Dade, Düdingen, Switzerland). Viability was >98% as assessed by staining with propidium iodide and annexin V-fluorescein isothiocyanate (FITC) (BD Biosciences, San Diego, Calif.).

The human EoL-3 eosinophilic leukemic cell line (Saito H, et al.) was from Richard Lynch (University of Iowa, Iowa City, Iowa) and cultured as described in Johansson M W, et al., 2004.

2. VCAM-1 Adhesion Assay

Wells in six-well culture plates (Corning, Corning, N.Y.) were coated with 1 ml of different concentrations of recombinant soluble extracellular 7-domain-VCAM-1⁴² in Tris-buffered saline (TBS) for 2 h at 37° C. (Johansson M W, et al. 2004) The VCAM-1-coated wells were washed with TBS, blocked with 0.1% gelatin (for eosinophils) or 1% BSA (for EoL-3 cells) in TBS for 30 minutes at 37° C., and washed with TBS. One ml of purified blood eosinophils (4×10⁶/ml) or EoL-3 cells (2×10⁶/ml) in RPMI 1640 with 0.2% BSA were plated in each well. After incubation for 1 h at 37° C., non-adherent cells were harvested, spun down for 10 minutes at 4° C. at 1,200 (eosinophils) or 1,500 (EoL-3 cells) revolutions per minute (rpm), resuspended in 400 μl FACS buffer (phosphate-buffered saline [PBS] with 2% BSA and 0.2% NaN₃), and processed for flow cytometry. The flow cytometry procedure was as described in Example 2 supra.

Results and Discussion

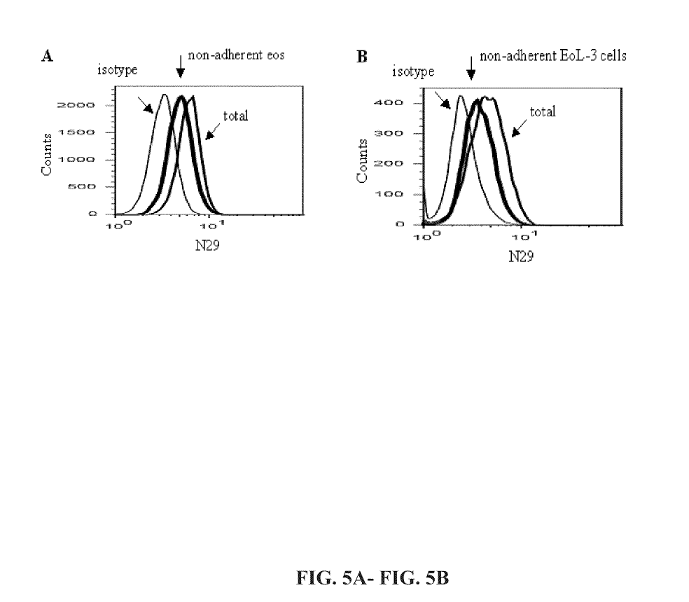

Purified blood eosinophils or EoL-3 leukemic eosinophilic cells were plated in VCAM-1-coated wells, and the cells that did not adhere to the substrate were compared to the starting population and to cells plated in wells coated only with control blocking protein. The leukemic cells were initially used to optimize the assay. The non-adherent eosinophils and EoL-3 cells had decreased N29 reactivity compared to the original population as demonstrated by the peak for N29 epitope expression being shifted to the left (FIG. 5A and FIG. 5B). Such a shift was not seen for total β₁ or β₂. Further, cells that did not adhere to the control substrate (gelatin for eosinophils and BSA for EoL-3 cells) had the same N29 epitope expression as the original population (data not shown). The non-adherent population had decreased N29 reactivity, indicating that VCAM-1 preferentially supports adhesion of those cells with a relatively higher N29 reactivity.

Overall, the data presented herein are compatible with a scenario that P-selectin; derived from plasma, activated platelets and/or endothelial cells; associates with eosinophils, enhances eosinophil β₁ activation state; stimulating eosinophil arrest on VCAM-1, egress and recruitment to the airway. P-selectin-triggered eosinophil β₁ activation is thus a potential therapeutic target.

Insights into the possible P-selectin-triggered intracellular signaling pathway(s) in eosinophils may be obtained from reports in the literature on PSGL-1-mediated signaling in neutrophils or leukemic cell lines, with endpoints other than integrin activation. In neutrophils, PSGL-1 ligation leads to protein tyrosine phosphorylation, activation of Ras activation, mitogen-activated protein kinase kinase (MEK) and extracellular-regulated kinase (ERK), redistribution of c-Abl; and leads to IL-8 secretion. (Hidari K I, et al.; Ba X, et al.) In HL-60 or U937 cells, the PSGL-1 cytoplasmic domain associates with Syk and PSGL-1 ligation leads to Syk tyrosine phosphorylation and serum response element-dependent transcription. (Urzaninqui A, et al.)

### Example 4

Further to our previous investigations, we investigated the correlation between β₁ integrin activation state and the level of P-selectin associated with eosinophils in whole blood obtained from participants in the multi-center Severe Asthma Research program (SARP). Participants in the SARP study form a heterogeneous population including mild/moderate asthmatics, severe asthmatics, and normal donors. β₁ integrin activation state was assessed with activation-sensitive anti-β₁ mAb N29. The blood obtained from a subset of subjects also was analyzed using anti-β-1 mAbs 8E3, 12G10, HUTS-21 and 9EG7.

Methods

1. Subjects

Blood samples from thirty-four (34) participants in the SARP study were analyzed (9 severe asthmatics, 20 mild/moderate asthmatics, and 5 normal donors). The American Thoracic Society consensus definition to determine severity classifications was used for inclusion in the SARP study (Sorkness et al. 2007). Inclusion criteria were as follows:

To qualify as a severe asthmatic, a history of physician-diagnosed asthma and asthma therapy for at least 6 months prior to screening was required. Severe asthmatics had to fulfill one of two major criteria: (1) continuous or near-continuous use of oral corticosteroids; or (2) use of high-dose ICS (880 μg/day or higher of fluticasone propionate or equivalent). They also had to meet two of seven minor criteria: (1) daily controller medication (in addition to ICS); (2) beta-agonist required daily or near-daily; (3) persistent airway obstruction (FEV₁<80% predicted, diurnal peak flow variability >20%); (4) one or more urgent care visits per year; (5) three or more oral corticosteroid bursts in the last 12 months; (6) prompt deterioration with 25% or greater reduction in oral or inhaled corticosteroid dose; and (7) a near-fatal asthma event in the past.

An individual could qualify as a mild/moderate asthmatic if the following criteria were met: a history of physician-diagnosed asthma and asthma therapy for at least 6 months prior to screening, a PC20 of 8 mg/ml or lower, and use of beta-agonist only or low-to-moderate dose of ICS (less than 880 μg/day of fluticasone propionate or equivalent), or not fulfilling the criteria for severe asthma.

To qualify as a normal donor, the following criteria were needed: a normal flow volume loop (by spirometry) with no indication of restrictive or obstructive airway disease, 5% or lower reversibility after albuterol, negative skin test, and PC20 of 25 mg/ml or higher.

Subjects had six-eight visits. All studies were approved by the University of Wisconsin-Madison Health Sciences Human Subjects Committee. Informed consent was obtained from each subject before participation.

2. Assessments

Clinical assessments were performed as previously described (Johansson M W et al. 2006). Spirometry for the determination of FEV₁ was carried out according to American Thoracic Society standards (Am J Respir Crit Care Med 1995; 152:1107-36) at four of the visits. Methacholine challenge to assess airway responsiveness was performed at one of the visits as previously described (Crapo R O et al.). Blood was drawn at two-three visits into CTAD (citrate, theophylline, adenosine, and dipyridamole) tubes (BD Vacutainer Systems, Franklin Lake, N.J.) and directly processed for flow cytometry.

3. Antibodies

Activation-sensitive anti-β₁ integrin mAbs 8E3 (Coe A P, et al.) and 12G10 (Mould A P, et al., 2005) were obtained from Martin Humphries (Wellcome Trust Center for Extracellular Matrix Research, Manchester, UK). Activation-sensitive anti-β₁ mAbs HUTS-21 (Luque A et al.) and 9EG7 (Bazzoni G, et al.); phycoerythrin (PE)-conjugated goat anti-mouse and anti-rat immunoglobulin (Ig) G; anti-P-selectin AC1.2; FITC-conjugated anti-CD14 and anti-CD16; and isotype controls mouse IgG₁, κ (clone A112-2), mouse IgG₂ₐ, κ (G155-178), and rat IgG₂ₐ, κ (A110-2) were from BD Biosciences. Activation-sensitive anti-β₁ N29 (Wilkins J A, et al.) were from Chemicon (Temecula, Calif.).

4. Flow Cytometry

Whole blood (100 μl) was incubated with 0.5 μg primary antibody or isotype control in 100 μl FACS buffer for 30 minutes. After primary antibody incubation, samples were washed with 1 ml PBS, washed with 250 μl FACS buffer, and resuspended in 250 μl FACS buffer containing PE-conjugated secondary antibody (2 μg/ml). After incubation for 30 minutes, samples were washed again with PBS, resuspended in 100 μl FACS buffer with FITC-conjugated anti-CD14 (0.125 μg) and anti-CD16 (0.625 μg) and incubated for 30 minutes. Red blood cells were lysed by incubation with 2 ml FACS lysing solution (BD Biosciences) for 10 minutes, followed by centrifugation. Incubations were at room temperature until after red blood cell lysis and then at 4° C. Samples were washed with 500 μl FACS buffer, resuspended in 250 μl FACS fixative (1% paraformaldehyde, 67.5 mM sodium cacodylate, 113 mM NaCl, pH 7.2), stored at 4° C. in the dark, and washed with 1 ml PBS and resuspended in 250 μl FACS buffer just prior to data collection. Fixation did not decrease signals (not shown).

Data were collected from 30,000-170,000 events with whole blood samples using a FACS Calibur (BD Biosciences; available through the Flow Cytometry Facility, Comprehensive Cancer Center, University of Wisconsin-Madison). Mid-range one-peak “rainbow” fluorescent beads (catalog No. RFP-30-5A, lot No. W02; Spherotech, Libertyville, Ill.) were first run at setup to check the optical alignment and set the sensitivity of the different detectors at a standardized fluorescence intensity, thus optimizing comparisons of data among subjects. Data were collected using Cellquest (BD Biosciences) and analyzed using FlowJo (TreeStar, Ashland, Oreg.). Post-data collection compensation for possible overlap between fluorochromes was performed using matrix algebra by FlowJo to remove any degree of subjectivity introduced by the manual compensation performed at setup. Eosinophils in whole blood were gated based both on scattering and lack of staining with anti-CD14 and anti-CD16, i.e., the cells that were analyzed for PE signal fit two criteria for eosinophils by being gated inside both characteristic regions in a plot of side scatter versus FITC staining and a plot of side versus forward scatter.

5. Statistics

The Spearman rank correlation test was used to analyze correlations. For subjects for which data were available from more than one visit, data from only one (selected by coin toss) of those visits were included in the correlation analysis. A level of probability (P)≦0.05 was considered significant. Analyses were performed using Prism 3.0 (GraphPad, San Diego, Calif.).

Results and Discussion

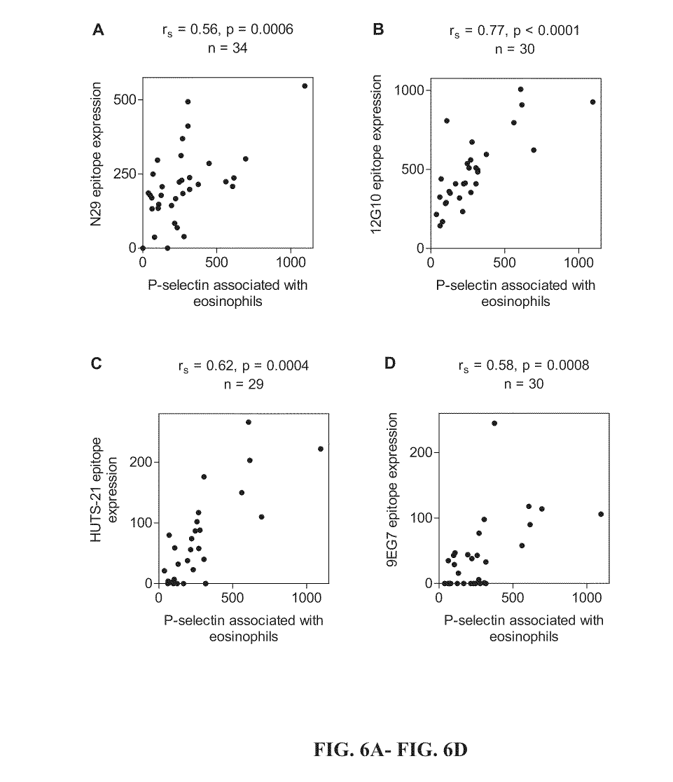

A significant correlation existed between β₁ integrin activation state, as assessed with activation-sensitive anti-β₁ mAb N29, and eosinphil-associated P-selectin in blood samples obtained from participants in the SARP study (see FIG. 6A). Further, this correlation was observed with other antibodies directed toward β₁ integrin. FIG. 6B reports the correlation between β₁ integrin activation state, as measured by anti-β₁ mAb 12G10, and eosinophil-associated P-selectin. Similarly, FIG. 6C reports the correlation between β₁ integrin activation state, as measured by anti-β₁ mAb HUTS-21 and eosinophil-associated P-selectin. Finally, FIG. 6D reports the correlation between β₁ integrin activation state, as measured by anti-β₁ mAb 9EG7, and eosinophil-associated P-selectin.

These data indicate that the binding of P-selectin to eosinophils causes enhanced β₁ integrin activation and exposure of the epitopes for the activation sensitive anti-β₁ integrin monoclonal antibodies. The activation may consist of coordinated changes in the mild/moderate asthmatics while more diverse changes may be observed in a heterogeneous population comprising mild/moderate asthmatics, severe asthmatics, and normal individuals.

Table 6 reports the correlation between the anti-β₁ integrin mAb N29 and other activation sensitive monoclonal antibodies on eosinophils in blood samples obtained from participants in the SARP study. The reactivity of anti-β₁ integrin mAb 8E3 is very similar to that of mAb N29 (see Table 6). The three other activation sensitive anti-β₁ integrin monoclonal antibodies, 12G10, HUTS-21, and 9EG7, also react similarly to N29 but to varying degrees (see Table 6). The data demonstrate that the correlations between β₁ integrin activation state and P-selectin and among β₁ integrin mAbs found in the Virus Induced Asthma Exacerbation (VIAX) study, discussed above, also exist in samples obtained from the heterogeneous population that exists in the SARP study. The r values obtained by analyzing samples from the SARP study range from 0.76 (8E3) to 0.43 (9EG7). These r values generally are lower than the values obtained by analyzing samples from the VIAX study (compare Table 5, which presents data obtained by analyzing samples from the VIAX study, to Table 6).

TABLE 6
Correlations between N29 epitope expression and expression of epitopes
for other activation-sensitive anti-pi integrin monoclonal antibodies
(mAbs) on eosinophils in blood samples from the SARP study

| β₁ integrin mAb | rₛ | p | n |
|---|---|---|---|
| 8E3 | 0.76 | <0.0001 | 30 |
| 12G10 | 0.45 | 0.01 | 30 |
| HUTS-21 | 0.48 | 0.009 | 29 |
| 9EG7 | 0.43 | 0.02 | 30 |

rₛ, Spearman rank correlation coefficient; P, probability; n, number of subjects.

It is understood that the various preferred embodiments are shown and described above to illustrate different possible features of the invention and the varying ways in which these features may be combined. Apart from combining the different features of the above embodiments in varying ways, other modifications are also considered to be within the scope of the invention. The invention is not intended to be limited to the preferred embodiments described above, but rather is intended to be limited only by the claims set out below.

### BIBLIOGRAPHY

Andre P. P-selectin in haemostasis. Br J Haematol 2004; 126:298-306.
Ba X, Chen C, Gao Y, Zeng X. Signaling function of PSGL-1 in neutrophil: tyrosine-phosphorylation-dependent and c-Abl-involved alteration in the F-actin-based cytoskeleton. J Cell Biochem 2005; 94:365-73.
Barthel S R, Jarjour N N, Mosher D F, Johansson M W. Dissection of the hyperadhesive phenotype of airway eosinophils in asthma. Am J Respir Cell Mol Biol 2006; 35:378-86.
Bazzoni G, Shib D-T, Buck C A, Hemler M E. Monoclonal antibody 9EG7 defines a novel β₁ integrin epitope induced by soluble ligand and manganese, but inhibited by calcium. J. Biol. Chem. 1995; 270:25570-7.
Coe A P, Askari J A, Kline A D, Robinson M K, Kirby H, Stephens P E, et al. Generation of a minimal alpha5beta1 integrin-Fc fragment. J Biol Chem 2001; 276:35854-66.
Crapo R O, Casaburi R, Coates A L, Enright P L, Hankinson J L, Irvin C G, et al. Guidelines for methacholine and exercise challenge testing-1999. This official statement of the American Thoracic Society was adopted by the ATS Board of Directors, July 1999. Am J Respir Crit Care Med 2000; 161:309-29.
da Costa Martins P A, van Gils J M, Mol A, Hordijk P L, Zwaginga J J. Platelet binding to monocytes increases the adhesive properties of monocytes by up-regulating the expression and functionality of beta1 and beta2 integrins. J Leukoc Biol 2006; 79:499-507.
Dransfield I, Hogg N. Regulated expression of Mg2+ binding epitope on leukocyte integrin alpha subunits. EMBO J 1989; 8:3759-65.
Gearing A J, Newman W. Circulating adhesion molecules in disease. Immunol Today 1993; 14:506-12.
Giembych M A, Lindsay M A. Pharmacology of the eosinophil. Pharmacol Rev 1999; 51:213-339
Hansel T T, De Vries I J, Iff T, Rihs S, Wandzilak M, Betz S, et al. An improved immunomagnetic procedure for the isolation of highly purified human blood eosinophils. J. Immunol. Methods 1991; 145:105-10.
Hidari K I, Weyrich A S, Zimmerman G A, McEver R P. Engagement of P-selectin glycoprotein ligand-1 enhances tyrosine phosphorylation and activates mitogen-activated protein kinases in human neutrophils. J Biol Chem 1997; 272:28750-6.
Humphries M J. Monoclonal antibodies as probes of integrin priming and activation. Biochem Soc Trans 2004; 32:407-11.
Humphries M J, Symonds E J, Mould A P. Mapping functional residues onto integrin crystal structures. Curr Opin Struct Biol 2003; 13:236-43.
Ikeda H, Nakayama H, Oda T, Kuwano K, Muraishi A, Sugi K, et al. Soluble form of P-selectin in patients with acute myocardial infarction. Coron Artery Dis 1994; 5:515-8.
Johansson M W, Barthel S R, Swenson C A, Evans M D, Jarjour N N, Mosher D F, et al. Eosinophil beta(1) integrin activation state correlates with asthma activity in a blind study of inhaled corticosteroid withdrawal. J Allergy Clin Immunol 2006; 117:1502-4.
Johansson M W, Lye M H, Barthel S R, Duffy A K, Annis D S, Mosher D F. Eosinophils adhere to vascular cell adhesion molecule-1 via podosomes. Am J Respir Cell Mol Biol 2004; 31:413-22.
Johansson M W, Kelly E A B, Busse W W, Jarjour N N, Mosher D F. Up-regulation and activation of eosinophils in blood and airway after segmental lung antigen challenge. J Immunol 2008; 180:7622-35.
Kowal K, Pampuch A, Kowal-Bielecka O, DuBuske L M, Bodzenta-Lukaszyk A. Platelet activation in allergic asthma patients during allergen challenge with Dermatophagoides pteronyssinus. Clin Exp Allergy 2006; 36:426-32.
Lu C, Shimaoka M, Zang Q, Takagi J, Springer T A. Locking in alternate conformations of the integrin alphaLbeta2 I domain with disulfide bonds reveals functional relationships among integrin domains. Proc Natl Acad Sci USA 2001; 98:2393-8.
Luo B H, Springer T A. Integrin structures and conformational signaling. Curr Opin Cell Biol 2006; 18:579-86.
Luque A, Gomez M, Puzon W, Takada Y, Sanchez-Madrid F, Cabanas C. Activated conformations of very late activation integrins detected by a group of antibodies (HUTS) specific for a novel regulatory region (355-425) of the common beta 1 chain. J Biol Chem 1996; 271:11067-75.
Mould A P, Garratt A N, Askari J A, Akiyama S K, Humphries M J. Identification of a novel anti-integrin monoclonal antibody that recognises a ligand-induced binding site epitope on the beta 1 subunit. FEBS Lett 1995; 363:118-22.
Mould A P, Travis M A, Barton S J, Hamilton J A, Askari J A, Craig S E, et al. Evidence that monoclonal antibodies directed against the integrin beta subunit plexin/semaphorin/integrin domain stimulate function by inducing receptor extension. J Biol Chem 2005; 280:4238-46.
Oymar K, Bjerknes R. Differential patterns of circulating adhesion molecules in children with bronchial asthma and acute bronchiolitis. Pediatr Allergy Immunol 1998; 9:73-9.
Palecek S P, Loftus J C, Ginsberg M H, Lauffenburger D A, Horwitz A F. Integrin-ligand binding properties govern cell migration speed through cell-substratum adhesiveness. Nature 1997; 385:537-40.
Saito H, Bourinbaiar A, Ginsburg M, Minato K, Ceresi E, Yamada K, et al. Establishment and characterization of a new human eosinophilic leukemia cell line. Blood 1985; 66:1233-40.
Seminario M C, Bochner B S. Expression and function of beta 1 integrins on human eosinophils. Mem Inst Oswaldo Cruz 1997; 92(Suppl 2):157-64.
Sjosward K N, Uppugunduri S, Schmekel B. Decreased serum levels of P-selectin and eosinophil cationic protein in patients with mild asthma after inhaled salbutamol. Respiration 2004; 71:241-5.
Sorkness R L, Bleecker E R, Busse W W, Calhoun W J, Castro M, Chung K F, Curran-Everett D, Erzurum S C, Gaston B M, Israel E, Jarjour N N, Moore W C, Peters S P, Teague W G, Wenzel S E. Lung function in adults with stable but severe asthma: air trapping and incomplete reversal of obstruction with bronchodilation. J Applied Physiol 2008; 104:394-403.
Standardization of Spirometry, 1994 Update. American Thoracic Society. Am J Respir Crit Care Med 1995; 152:1107-36.
Urzainqui A, Serrador J M, Viedma F, Yanez-Mo M, Rodriguez A, Corbi A L, et al. ITAM-based interaction of ERM proteins with Syk mediates signaling by the leukocyte adhesion receptor PSGL-1. Immunity 2002; 17:401-12
Weller P F, Rand T H, Goelz S E, Chi-Rosso G, Lobb R R. Human eosinophil adherence to vascular endothelium mediated by binding to vascular cell adhesion molecule 1 and endothelial leukocyte adhesion molecule 1. Proc Natl Acad Sci USA 1991; 88:7430-3.
Wilkins J A, Li A, Ni H, Stupack D G, Shen C. Control of beta1 integrin function. Localization of stimulatory epitopes. J Biol Chem 1996; 271:3046-51.
Xu D Y, Zhao S P, Peng W P. Elevated plasma levels of soluble P-selectin in patients with acute myocardial infarction and unstable angina. An inverse link to lipoprotein(a). Int J Cardiol 1998; 64:253-8.
Yago T, Tsukuda M, Minami M. P-selectin binding promotes the adhesion of monocytes to VCAM-1 under flow conditions. J Immunol 1999; 163:367-73.
Zhu X, Munoz N M, Kim K P, Sano H, Cho W, Leff A R. Cytosolic phospholipase A2 activation is essential for beta 1 and beta 2 integrin-dependent adhesion of human eosinophils. J Immunol 1999; 163:3423-9.

What is claimed is:

1. A method for detecting P-selectin associated with eosinophils, the method comprising detecting an amount of P-selectin associated eosinophils in a sample from a subject and quantifying the total number of eosinophils in the sample.

2. The method of claim 1, wherein the step of detecting comprises conducting an assay to determine binding of P-selectin to a binding partner of P-selectin

3. The method of claim 2, wherein the step of conducting an assay is chosen from at least one of flow cytometry, an ELISA assay, and an ELISA-type assay.

4. The method of claim 2, wherein the binding partner is an antibody that binds to P-selectin.

5. The method of claim 1, further comprising:
(a) determining a baseline of P-selectin in a baseline sample from the subject;
(b) detecting an amount of P-selectin in an additional sample from the subject; and
(c) comparing the baseline amount of P-selectin to the amount of P-selectin in the additional sample.

6. A method for monitoring asthma, the method comprising:
(a) detecting an amount of P-selectin in a sample from a subject; and
(b) comparing the amount of P-selectin to a baseline amount, wherein an increase or decrease in the amount of P-selectin is indicative of a change in asthmatic condition.

7. The method of claim 6, wherein the step of detecting comprises conducting an assay to determine binding of P-selectin to a binding partner of P-selectin

8. The method of claim 7, wherein the step of conducting an assay is chosen from at least one of flow cytometry, an ELISA assay, and an ELISA-like assay.

9. The method of claim 7, wherein the binding partner is an antibody that binds to P-selectin.

10. The method of claim 6, wherein the method further comprising detecting an amount of at least partially activated β-1 integrin.

11. The method of claim 10, wherein the step of detecting comprises conducting an assay to determine binding of at least partially activated β-1 integrin to a binding partner of activated β-1 integrin.

12. The method of claim 11, wherein the step of conducting an assay is selected from the group consisting of flow cytometry, an ELISA assay, and an ELISA-type assay.

13. The method of claim 12, wherein the binding partner is an antibody that binds to at least partially activated β-1 integrin.

14. The method of claim 13, wherein the antibody is selected from the group consisting of: mAb N29, mAb 8E3, mAb HUTS-21, and mAb 9EG7.

15. The method of claim 14, wherein the antibody is mAb N29.

16. A kit for determining relative amount of P-selectin in a subject, the kit comprising:
(a) a positive control comprising cells that express P-selectin;
(b) a negative control comprising cells lacking P-selectin; and
(c) instructions for how to use the kit.

17. The kit of claim 16, wherein the kit further comprises an antibody that binds P-selectin.

18. A method of monitoring a biological condition, the method comprising:
(a) detecting an amount of P-selectin in a sample from a subject;
(b) detecting an amount of β-1 integrin activation; and
(c) comparing the amount of P-selectin and β-1 integrin activation to a baseline amount, wherein an increase or decrease in the amount of P-selectin and β-1 integrin activation is indicative of a change in a biological condition.

19. The method of claim 18, wherein the biological condition is asthma.

20. The method of claim 18 wherein the step of detecting P-selectin comprises conducting an assay to determine binding of P-selectin to a binding partner of P-selectin
